(19) United States
(12) Patent Application Publication
(10) Pub. No.: US 2004/0229217 A1
(43) Pub. Date: Nov. 18, 2004

(54) Screening process for various indications using BNPI and/or DNPI

(75) Inventors: Eberhard Weihe, Marburg (DE); Martin K-H Schaefer, Marburg (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/807,500

(22) Filed: Mar. 24, 2004

Related U.S. Application Data
(63) Continuation of application No. PCT/EP02/10707, filed on Sep. 24, 2002.

(30) Foreign Application Priority Data
Sep. 24, 2001 (DE) 101 47 006.1
Sep. 25, 2001 (DE) 101 47 028.2

(51) Int. Cl. C12Q 1/70; C12Q 1/68

(52) U.S. Cl. 435/5; 435/6

(57) ABSTRACT

Methods for finding and screening for pharmaceutically active substances using BNPI and/or DNPI or biomolecules derived therefrom, and compounds identified thereby and their use. Also provided are active substances that bind to BNPI and/or DNPI, antibodies directed against BNPI and/or DNPI, antisense nucleotides against BNPI and/or DNPI, or BNPI and/or DNPI or partial proteins thereof, or of corresponding polynucleotides, in the preparation of pharmaceutical formulations for the treatment of various diseases or for related methods of treatment and diagnostics.

### CROSS-REFERENCE TO RELATED APPLICATIONS

This application is a continuation of International Patent Application No. PCT/EP02/10707, filed Sep. 24, 2002, designating the United States of America, and published in German as WO 03/029828, the entire disclosure of which is incorporated herein by reference. Priority is claimed based on Federal Republic of Germany Patent Application Nos. DE 101 47 006.1, filed on Sep. 24, 2001, and DE 101 47 028.2, filed on Sep. 25, 2001.

### FIELD OF THE INVENTION

The invention relates to a process for finding pharmaceutically active substances using BNPI and/or DNPI or biomolecules derived therefrom, and to the use of compounds identified thereby, of active substances that bind to BNPI and/or DNPI, of antibodies directed against BNPI and/or DNPI, of antisense nucleotides against BNPI and/or DNPI, or of BNPI and/or DNPI or partial proteins thereof, or of corresponding polynucleotides, in the preparation of pharmaceutical formulations for the treatment of various diseases or for treatment methods and diagnostics.

### BACKGROUND OF THE INVENTION

The finding of the sites of action of pharmaceutical active substances, the so-called targets, is one of the most important tasks of modern pharmaceutical research. Via the affinities for these targets, or via the physiological effects induced by interaction with these targets, it is possible, by means of so-called “screening methods”, to filter from the large number of known substances, for example from the substance libraries of pharmaceutical research, valuable substances or classes of substance which in all probability are active in the indications associated with this target. The most important representatives of these targets include proteins, generally receptors, especially G-protein-coupled receptors, and transport proteins. However, such targets are sometimes very difficult to find because the potential choice is very large. For orientation and identification there are used on the one hand knowledge about the (physiological) function with the (potential) position in signal cascades and metabolic pathways, but on the other hand also localization and degree of expression in the various tissues. Within the scope of this invention, particular attention has been given to the central nervous system, where not only a general localization but also a very specific and precise distribution in the various regions are important.

### SUMMARY OF THE INVENTION

Accordingly, one object of the invention was to find and identify one or more such targets, especially having localization and activity in the central nervous system, and to develop a corresponding screening process. Accordingly, the invention relates to a process for finding pharmaceutically relevant substances that are active in the following indications or for the treatment of

visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischemic attacks), emesis, especially hyperemesis, for example during chemotherapy, dizziness in any form, cataracts, arthritis, hyperactivity, developmental disorders, rabies, viral infections or bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, especially glutamate-mediated oversensitivity, neurodegeneration especially in Alzheimer's disease, Alzheimer's disease, ischemia; encephalitis, especially viral or bacterial encephalitis; prion disease, Rasmussen's encephalitis, HIV encephalitis, demyelinisation especially in multiple sclerosis, retinal degeneration, glaucoma, nystagmus, detachment of the retina, diseases of the cerebellum, cerebellar ataxia, diseases of the basal ganglia, diseases of the pallidum, diseases of the organ of hearing and/or balance, diseases of the auditory canal or vestibular canal, memory disorders, learning disorders, cognitive disorders, stiff man syndrome, restless leg syndrome, anxiety, phobias, sleep disorders; drug dependency, addiction and withdrawal, especially in the case of alcohol, nicotine, opiates, Ecstasy or cocaine; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, such as impotence or priapism, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease, comprising the following process steps:

(a) incubation of a substance to be tested under suitable conditions with at least one biomolecule of group I selected from:

the protein BNPI and/or DNPI and/or a protein according to one of FIGS. 1b) (SEQ ID NO:2), 1d) (SEQ ID NO:4), 1f) (SEQ ID NO:6), 1h) (SEQ ID NO:8), 2b) (SEQ ID NO: 10), 2d) (SEQ ID NO:12) or 2f) (SEQ ID NO:14)and/or a protein that is at least 90% similar to one of the above-mentioned proteins and/or a protein coded for by a polynucleotide according to one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO: 13) or by a polynucleotide that is at least 90% similar thereto, and/or a protein coded for by a nucleic acid that binds under stringent conditions to a polynucleotide according to one of FIGS. 1a) (SEQ ID NO: 1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or their antisense polynucleotides, or a partial protein of one of the above-mentioned proteins that has a length of at least 10, preferably at least 15, especially at least 20 amino acids, and/or a cell and/or a preparation of such a cell that has synthesized at least one of the above-mentioned proteins and partial proteins, or biomolecules of group I,

(b) measurement of the binding of the test substance to the biomolecule(s) of group I optionally synthesized by a cell or to a cell and/or a preparation of such a cell that has synthesized at least one of the above-mentioned biomolecules of group I, or measurement of at least one functional parameter changed by the binding of the test substance to the biomolecule(s) of group I optionally synthesized by a cell or to a cell and/or a preparation of such a cell that has synthesized at least one of the above-mentioned biomolecules of group I.

This novel screening process is based on the fact that a potential medicinal activity of a substance can be found via its interaction with at least one physiologically relevant protein or peptide structure, a target, BNPI and/or DNPI or related structures. In the literature and in some cases also in this invention, BNPI is referred to as VGLUT1 and DNPI as VGLUT2. Within the scope of this invention in particular, these terms are therefore to be regarded as wholly synonymous. BNPI and DNPI, or the proteins and partial proteins or peptides derived therefrom and listed herein, or nucleic acids coding therefor, have been identified as targets of interest within the scope of this invention. BNPI and DNPI exhibited localization in very different regions of the CNS, but surprisingly—despite in some cases the closest proximity—always strictly separate localization, this strict separation clearly indicating that important physiological processes are controlled by DNPI and BNPI. BNPI (VGLUT1) is strongly expressed especially by a population of large DRG neurons, which are substance-P-negative. DNPI (VGLUT2), by contrast, mRNA is found especially in the medium-sized and small substance-P-positive neurons. The localization of VGLUT1 (BNPI) and VGLUT2 (DNPI) is wholly independent of the localization of the related transporter VGLUT3. Because DNPI and BNPI are localized in regions of the CNS that are of great interest therapeutically and therefore are of interest for a corresponding large number of indications, DNPI and BNPI are correspondingly important targets with which screening processes can be carried out on pharmacologically active compounds. Consequently, it is also preferred if BNPI and DNPI, or in each case one of the biomolecules derived therefrom and listed herein, such as proteins and partial proteins or peptides or a nucleic acid coding therefor, are used simultaneously in a screening process, or the result of two separate screening processes on the one hand using BNPI or one of the biomolecules derived therefrom and listed herein, such as protein and partial protein or peptide or a nucleic acid coding therefor, and on the other hand using DNPI or one of the biomolecules derived therefrom and listed herein, such as protein and partial protein or peptide or a nucleic acid coding therefor, are carried out and pharmacologically active substances are identified in both cases by differential comparison of the data, which substances are then highly specific.

The terms pharmaceutically relevant or pharmacologically active relate to a potentially healing or alleviating influence of the substance on a particular disease pattern. The term substance includes any compound suitable as a pharmacological active substance, especially therefore low molecular weight active substances, but also other substances such as nucleic acids, fats, sugars, peptides or proteins such as antibodies.

Incubation under suitable conditions is here to be understood as meaning that the substance to be tested is able to react with the biomolecule or the cell or the corresponding preparation in an aqueous medium for a defined time before the measurement. The aqueous medium may be heated, for example at from 4° C. to 40° C., preferably at room temperature or at 37° C. The incubation time can be varied from a few seconds to several hours depending on the interaction of the substance with the biomolecule or partial protein or protein. However, times from 1 minute to 60 minutes are preferred. The aqueous medium may contain suitable salts and/or buffer systems so that, for example, a pH from 6 to 8, preferably pH 7.0 to 7.5, prevails in the medium during the incubation. Further suitable substances, such as coenzymes, nutrients, etc., can be added to the medium. The suitable conditions can readily be determined by the person skilled in the art, depending on the interaction to be studied of the substance with the partial protein or protein, on the basis of his experience, the literature or a small number of simple preliminary experiments, in order to obtain as clear a measured value as possible in the process.

A cell that has synthesized a particular partial protein or protein or biomolecule is a cell that has already expressed that partial protein or protein endogenously or a cell that has been modified by genetic engineering so that it expresses the partial protein or protein or biomolecule and accordingly contains the partial protein or protein before the beginning of the process according to the invention. The cells may be cells from optionally immortalized cell lines or native cells originating from tissues and isolated therefrom, the cell structure in most cases being dissolved. The preparation of those cells comprises especially homogenates from the cells, the cytosol, a membrane fraction of the cells with membrane fragments, a suspension of isolated cell organelles, etc.

It is unimportant for the working of the process from which species the proteins, partial proteins or biomolecules originate, but it is preferred to use the human, mouse or rat variant. BNPI and DNPI are known in respect of the coding DNA sequence and the amino acid sequence and their general function has also been described. BNPI, the brain Na+dependent inorganic phosphate cotransporter, is described in WO 96/34288, and DNPI, the differentiation-associated Na+dependent inorganic phosphate cotransporter, has been described by Aihara et al. (2000) in J. Neurochem. 74, 2622-2625. In addition to its function as the sodium-dependent phosphate transporter, a vesicular glutamate transporter function has also been described for BNPI and BNPI has been designated VGlutT1 (Bellocchio et al. (2000), Science 189:957-960; Takamori et al. (2000), Nature 407; 189-194).

The measure by which the process enables substances of interest to be found is either the binding to the biomolecule, the protein or partial protein, which can be detected, for example, by displacement of a known ligand or the amount of bonded substance, or the change in a functional parameter as a result of the interaction of the substance with the partial protein or protein or biomolecule. Such interaction may especially be the regulation, inhibition and/or activation of receptors, ion channels and/or enzymes, and changed functional parameters may be, for example, the gene expression, the ionic environment, the pH or the membrane potential, or a change in the enzyme activity or in the concentration of the 2nd messenger.

In order to explain the invention, in addition to the explanations of terms given in the general text, further definitions are given hereinbelow in order to clarify how particular terms, especially terms used in the claims, are to be understood and interpreted within the scope of this invention:

substance: This means a chemical compound. It relates in the narrow sense to compounds which are potentially able to develop an action in the body, low molecular weight active substances, nucleic acids, fats, sugars, peptides or proteins, in particular here low molecular weight active substances.

pharmaceutically relevant substance: Within the scope of the invention, a pharmaceutically relevant substance is a substance which, by binding to the biomolecules of groups I to III, could be active in at least one of the mentioned indications and theoretically has the potential physiologically to influence the symptoms directly or indirectly, especially a substance which appears capable of use therapeutically, for example in a medicament.

pain-regulating: Within the scope of the invention, pain-regulating means that the substance influences the perception of pain directly or indirectly, especially, but not exclusively, those having a natural analgesic action.

pain: Within the scope of the invention, pain means in particular a feeling of pain, more precisely of acute, chronic, neuropathic and inflammatory pain including migraine, the pain belonging in particular to the following types: chronic pain, especially musculoskeletal pain; neuropathic pain, especially allodynic pain, mechanical hyperalgesia or diabetic neuropathy; visceral pain, cerebral pain, peripheral pain or inflammation-related pain, especially peripheral inflammation-related pain; as well as migraine, cluster headache or the pain of trigeminal neuralgia.

incubation: Incubation is to be understood as meaning the introduction and leaving of a biological test object, for example a cell or a protein, in a heated medium, such as an incubation cabinet or a water bath. Under suitable conditions means incubation under physiological conditions (e.g., 37° C., pH 7.2) or under conditions under which an optimum measurement in the process is possible.

cell: The cell is a self-regulating, open system with its own metabolism which is in equilibrium of flow with its surroundings owing to permanent exchange of material and which is capable of multiplication. The cell can be cultivated separately or can be part of a tissue, especially from an organ, and may be isolated therefrom or still present in the cell structure.

preparation of a cell: This is understood to mean preparations that are prepared by means of chemical, biological, mechanical or physical methods with a change in the cell structure, for example membrane fragments, isolated cell compartments, isolated cytosol, or homogenate obtained from tissue.

peptide: Compound comprising amino acids linked via peptide bonds. An oligopeptide consists of from 2 to 9 amino acids, a polypeptide of from 10 to 100 amino acids.

protein: Compound comprising more than 100 amino acids linked via peptide bonds to form chains, sometimes with a defined spatial structure.

partial protein: Compound comprising more than 10 amino acids linked via peptide bonds to form chains, sometimes having a defined spatial structure, but cut out or selected from a defined protein. A partial protein may be a peptide.

PIM1 kinase; PIM3 kinase: A proto-oncogen and a serine-threonine kinase.

polynucleotide: The underlying nucleotide is a basic constituent of the nucleic acids consisting in principle of nucleic base, pentose and phosphoric acid. This corresponds to a high molecular weight polynucleotide of a plurality of nucleotides linked together via phosphoric acid-pentose esterification. However, this invention also includes modified polynucleotides, which retain the base sequence but have a modified backbone instead of phosphoric acid-pentose.

at least 90 (95, 97) % similar: This is to be understood as meaning that the polynucleotides so described are at least 90% (95%, 97%) identical in their coding region with the reference (figure, etc.) in respect of the base sequence, and the peptides and proteins so described are at least 90% (95%, 97%) identical with the reference in their primary structure, the sequence of the amino acids.

gene: The term gene denotes a genome section having a defined nucleotide sequence which contains the information for the synthesis of a m- or pre-m-RNA or another RNA (e.g., tRNA, rRNA, snRNA, etc.) It consists of coding and non-coding sections.

gene fragment: Nucleic acid section which contains a partial region of a gene in its base sequence.

biomolecule: General term for nucleic acids or polyamino acids, especially also DNA, RNA, peptides (partial proteins) and proteins. These molecules may be synthetically modified. Within the scope of this invention, peptides (partial proteins) and proteins are preferred

binding to the peptide, partial protein or protein: Interaction between a substance and peptide, partial protein or protein, leading to fixing.

functional parameters: These are measured values of an experiment which correlate with the function of a protein (such proteins may relate to an ion channel, receptor, enzyme).

manipulated by genetic engineering: Manipulation of cells, tissues or organisms in such a manner that genetic material is introduced.

expressed endogenously: Expression of a protein which contains a cell line under suitable culturing conditions, without the protein having been made to express by manipulation by genetic engineering.

G-protein: Internationally customary abbreviation for a guanosine triphosphate (GTP)-binding protein, which, as a signal protein, is activated by G-protein-coupled receptors.

reporter gene: General name for genes whose gene products can readily be detected with the aid of simple biochemical methods or histochemical methods, for example, luciferase, alkaline phosphatase or green fluorescent protein (GFP).

(recombinant) DNA construct: General name for any type of DNA molecules formed by the in vitro linking of DNA molecules.

cloning vector: General name for nucleic acid molecules which, in cloning, serve as carriers of foreign genes or parts of such genes.

expression vector: Name for specially constructed cloning vectors which, after introduction into a suitable host cell, permit the transcription and translation of the foreign gene cloned into the vector.

LTR sequence: Abbreviation for long terminal repeat. General name for longer sequence regions which are to be found at both ends of a linear genome. Such sequence regions occur, for example, in the genomes of retroviruses and at the ends of eukaryotic transposons.

poly-A tail: The adenyl radicals (about 20 to 250) attached to the 3′ end of messenger RNA's by polyadenylation.

promoter sequence: Name for a DNA sequencing region by which the transcription of a gene, i.e. the synthesis of the mRNA, is controlled.

ORI sequence: Abbreviation for origin of replication. The ORI sequence allows a DNA molecule to multiply as an autonomous unit in the cell.

enhancer sequence: Name for relatively short genetic elements, in some cases occurring as repetitions, which generally amplify the expression of some genes to differing degrees.

transcription factor: Name for a protein which, by bonding to specific DNA sequences, influences the transcription of a gene.

cultivate: To keep cells or tissue under suitable culturing conditions.

conditions permitting expression: This is understood to mean the choice and use of culturing conditions which permit expression of the protein in question, including temperature change, change of medium, addition of inducing substances, omission of inhibiting substances.

incubation time: Period of time for which cells or tissue are incubated, i.e. exposed to a defined temperature.

selection pressure: Use of culturing conditions that give cells having a particular gene product, the so-called selection marker, an advantage in terms of growth.

amphibian cell: Cell from an animal from the class of the Amphibia.

bacterial cell: Cell which is to be assigned to the order of the Eubacteria or Archaebacteria or which originates therefrom.

yeast cell: Cell which is to be assigned to the order of the Endomycetalse or which originates therefrom.

insect cell: Cell which is to be assigned to the order of the Hexapoda or which originates therefrom.

native mammalian cell: Cell originating from a mammal, which corresponds in its relevant features to the cell found in the organism.

immortalized mammalian cell: Cell which, as a result of the applied culturing conditions or manipulation by genetic engineering, has acquired the property of dividing in the culture beyond the normally usual frequency of division (about 100).

labeled: Made accessible for a detection reaction by appropriate modification or derivatization, for example, radioactive, fluorescent or luminescent.

ligand: Substance which binds to a molecule located in the body or in a cell, specifically a receptor.

displacement: Complete or partial removal of a ligand from its binding site.

bonded activity: measured value determined biochemically or physically, which correlates with the amount of ligand bonded to a receptor.

regulation: The inhibition or activation of an operation carried out as part of a regulating process.

inhibition: As a special case of regulation, the prevention/diminution of an operation.

activation: As a special case of regulation, the amplification of an operation.

receptors: In one sense, all molecules present in the prokaryotic or eukaryotic organism to which an active substance is able to bind. In another sense, membrane-bonded proteins or complexes of several proteins which, by binding an active substance, bring about a change in the cell.

ion channels: Membrane-bonded proteins or complexes of several proteins through which cations or anions are able to pass through the membrane.

enzymes: Name for proteins or complexes of an activating non-albuminous component with a protein, which possess catalytic properties.

gene expression (express/expressable): The translation of the genetic information of a gene into RNA (RNA expression) or into protein (protein expression).

ionic environment: Ion concentration of one or more ions in a particular compartment.

membrane potential: Potential difference across a membrane owing to an excess of cations on one side and anions on the other side of the membrane.

change in enzyme activity: Inhibition or induction of the catalytic activity of an enzyme.

2nd messenger: Small molecule which, in response to an extracellular signal, is either formed in the cytosol or migrates into the cytosol and aids the transmission of the information to the cell line, such as, for example, cAMP, IP₃.

(gene) probe: Name for any type of nucleic acids with the aid of which a desired gene or a particular DNA sequence can be detected. By derivatization of the gene probe (e.g., biotin, magnetic beads, digoxinin), it is additionally possible to extract DNA molecules from a mixture. Suitable probes may include cloned genes, gene fragments, chemically synthesized oligonucleotides and also RNA, which in most cases is radioactively labeled.

DNA: International name for deoxyribonucleic acid.

genomic DNA: General name for the DNA originating from the cell nucleus of a cell in eukaryotic organisms.

cDNA: Abbreviation for complementary DNA. Name for the single- or double-stranded DNA copy of an RNA molecule.

cDNA bank/library: Name for a collection of randomly cloned cDNA fragments which, taken together, represent the totality of all the RNA synthesized by a cell or a tissue.

cDNA clone: Name for a population of genetically uniform cells which derive from a single cell, so that this cell contains an artificially introduced cDNA fragment.

hybridization: Formation of a double-stranded nucleic acid molecule from two separate single stands by base pairing.

stringent conditions: Conditions under which only perfectly base-paired nucleic acid strands are formed and remain stable.

isolate: To find a desired molecule in a mixture and separate it therefrom.

DNA sequencing: Determination of the sequence of the bases in a DNA molecule.

nucleic acid sequence: Name for the primary structure of a DNA molecule, i.e. the sequence of the individual bases of which a DNA is composed.

gene-specific oligonucleotide primer: Oligonucleic acids, that is to say nucleic acid fragments having a length of from 10 to 40 bases, which in their base composition permit a stringent hybridization on the desired gene or the desired cDNA.

determination of oligonucleotide primers: The manual or computer-assisted search for oligonucleotides in a given DNA sequence which are optimally suitable for hybridization and/or a polymerase chain reaction.

PCR: Abbreviation for polymerase chain reaction. In vitro process for the selective concentration of nucleic acid regions of defined length and defined sequence from a mixture of nucleic acid molecules.

DNA template: Nucleic acid molecule or a mixture of nucleic acid molecules from which a DNA section is duplicated with the aid of the PCR (see above).

RNA: Internationally customary abbreviation for ribonucleic acids.

mRNA: Internationally customary abbreviation for messenger ribonucleic acids, which are involved in the transfer of the genetic information from the nucleus into the cell and contain information for the synthesis of a polypeptide or a protein.

antisense polynucleotide: A molecule consisting of a plurality of natural or modified nucleic acids, the base sequence of which molecule is complementary to the base sequence of a partial region of an RNA occurring in nature.

PNA: Internationally customary abbreviation for peptidic nucleic acids. Amino acids linked by peptides form a chain, the amino acids carrying as side chain a base capable of hybridization with DNA or RNA.

sequence: Sequence of nucleotides or amino acids. In the specific sense of this invention, it refers to the nucleic acid sequence.

ribozyme: Name for a catalytically active ribonucleic acid (e.g., ligase, endonuclease, polymerase, exonuclease).

DNA enzyme: Name for a DNA molecule which contains catalytic activity (e.g., ligase, endonuclease, polymerase, exonuclease).

catalytic RNA/DNA: General name for ribozymes or DNA enzymes (see above).

adenovirus: Cytopathogenic virus occurring in vertebrates.

adeno-associated virus (AAV): Belongs to the family of the paroviruses. For effective multiplication of the AAV, co-infection of the host cells with helper viruses (e.g., herpes viruses, vaccinia viruses or adenoviruses) is necessary. The property of AAV of integrating stably into the host genome makes it particularly valuable as a transduction vector for mammalian cells.

herpes virus: Viral pathogen of the herpes infection.

post-translational modification: Change carried out on proteins or polypeptides after translation, including, for example, phosphorylation, glycosylation, amidation, acetylation or proteolysis.

glycosylate: Name for the attachment of individual sugar molecules or entire sugar chains to proteins.

phosphorylate: Name for the attachment of one or more phosphate radicals to a protein, preferably to the OH groups of the amino acids serine, threonine or tyrosine.

amidate: The name for the conversion of a carboxyl function into an amide function, e.g., at the carboxy-terminal amino acid radical of a peptide or protein.

provided with membrane anchor: Post-translational modification of a protein, or of another organic molecule, in such a manner that it is anchored to the lipid double layer membrane of cells by attachment of a hydrophobic molecule, expediently a fatty acid or a derivative thereof.

cleave: In this specific case, the cleavage of a peptide or protein into several sub-sequences.

shorten: To shorten a molecule consisting of a plurality of individual parts by one or more parts.

antibodies: Proteins, referred to as immunoglobulins, which are soluble, or bonded to cell membranes, and have a specific binding site for antigens.

monoclonal antibody: Antibodies which are directed against a single antigenic determinant of an antigen and have extremely high selectivity.

polyclonal antibody: Mixture of antibodies directed against several determinants of an antigen.

transgenic: Genetically altered.

non-human mammal: The totality of the mammals (class of the Mammalia) with the exception of the species man.

germ cell: Cell having a haploid genome which, by fusion with a second germ cell, enables a new organism to form.

somatic cell: Diploid cell as constituent of an organism.

chromosomal incorporation: Insertion into the nucleotide sequence at chromosome level.

genome: General description for the totality of all the genes in an organism.

ancestor of the animal: An animal (the ancestor) which, naturally or artificially, is related with another animal (the descendant) in direct line by passing on of its genetic material.

expressable: A nucleic acid molecule is expressable when it contains the information for the synthesis of a protein or polypeptide and is provided with corresponding regulatory sequences which permit synthesis of that protein or polypeptide in vitro or in vivo. When those requirements are no longer met, for example as a result of interference in the coding sequence, then the nucleic acid molecule is no longer expressable.

rodent: Animal from the order of the Rodentia, e.g., rat or mouse.

identifiable as a pain-regulating substance: Substance which, when introduced into a living organism, brings about a behavioral change which the person skilled in the art describes as pain-inhibiting (antinociceptive, antihyperalgesic or antiallodynic). In the case of the screening process, this refers to the fact that, during screening, the substance markedly, for example 100%, exceeds the binding or interaction of the average of the tested substances by more pronounced binding or triggering of a change in a functional parameter.

compound: Another name for a molecule, as consisting of a plurality of atoms, here a molecule identified by the process according to the invention.

active substance: A compound which, when administered to an organism, brings about a change in the organism. It is understood in particular to mean organochemically synthesized molecules which have a healing effect on the organism. In the present case, especially molecules which bind to the proteins and peptides according to the invention.

low molecular weight: Molecule having a molecular weight <2 kDa.

medicament: A substance according to the definition in Article 1 §2 of the law on trade in medicaments.

diagnostic: A compound or process which can be used to diagnose a disease.

treatment of pain: Process aimed at alleviating or removing pain, or inhibiting the expected occurrence of pain (pre-emptive analgesia).

chronic pain: A feeling of pain of longer duration, often characterized by the fact that it extends beyond the time and site of the initial stimulus, the sensitivity of the body to pain increases.

gene therapy: Gene therapy is understood to mean all processes which aim to treat genetic diseases causally by suitable changes to the genome.

in vivo gene therapy: Introduction of genetic material into the living organism with the aim of gene therapy. A distinction can be made between somatic intervention and germ path intervention, the one being carried out on diploid cells and the other on haploid cells.

in vitro gene therapy: Introduction of genetic material into cells outside the human body with the aim of subsequently using the cells again for gene therapy by introducing them into the human body.

diagnostics: Process for identifying a disease.

activity study: Study with the aim of studying the activity of a compound after action on a living organism.

In a preferred embodiment of the process, the cell is manipulated by genetic engineering before step (a). Genetic material, especially one or more polynucleotide sequences, is thereby introduced into the cell. In a further preferred variant of this embodiment, the manipulation by genetic engineering allows at least one of the functional parameters changed by the test substance to be measured. In this embodiment, manipulation by genetic engineering creates conditions under which the change in a functional parameter can be measured or can be measured in an improved manner. It is particularly preferred for the manipulation by genetic engineering to effect the introduction of a form of a G-protein that is not endogenously expressed in the cell or the introduction of a reporter gene. This is to be understood as being especially the introduction into the cell by genetic engineering of a G-protein (GTP-binding protein) that is not present endogenously or not expressed physiologically, for example the introduction of a chimeric G-protein that permits a change of the signal pathway or of a promiscuitive G-protein that is very ready to bind. The introduction of a reporter gene in turn permits the measurement of an (extracellularly triggered) induced expression of the gene product.

In a further preferred embodiment, the cell is manipulated by genetic engineering in such a manner that the cell contains at least one polynucleotide according to one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or a polynucleotide that is at least 90%, preferably at least 95%, especially at least 97%, similar thereto. In that manner it is possible, for example, to make the cell synthesize a partial protein or protein that is not expressed endogenously in the cell or preparation used in the process. It is particularly preferred for the polynucleotide to be contained in a recombinant DNA construct. A (recombinant) DNA construct is understood to be a DNA molecule produced in vitro.

If in the process the cell is manipulated by genetic engineering before step (a), it is preferred for the cell to be cultivated, after the manipulation by genetic engineering and before step (a), under conditions permitting expression, optionally under selection pressure. Cultivation is understood to mean keeping cells or tissue under conditions which ensure survival of the cells or their succeeding generation. The conditions should be so chosen that expression of the material inserted by the manipulation by genetic engineering is made possible. To that end, the pH, oxygen content and temperature should be kept at physiological values, and adequate nutrients and necessary co-factors should be added. The selection pressure makes it possible to cultivate further only those cells in which the manipulation by genetic engineering was at least partly successful. This includes, for example, the introduction of antibiotic resistance via the DNA construct.

It is particularly preferred in the process according to the invention for the cell used to be an amphibian cell, bacterial cell, yeast cell, insect cell or an immortalized or native mammalian cell. Examples of amphibian cells are Xenopus oocytes, examples of bacterial cells are E. coli cells, examples of yeast cells are Saccharomyces cerevisiae, examples of insect cells are Sf9 cells, examples of immortalized mammalian cells are HeLa cells and examples of native mammalian cells are the CHO (Chinese hamster ovary) cell.

In a preferred measuring method for determining the binding of the substance to the biomolecule or partial protein or protein in the process according to the invention, the measurement of the binding is carried out via the displacement of a known labeled ligand of the biomolecule or of the partial protein or protein and/or via the activity of a labeled test substance bonded thereto. A ligand is a molecule that binds with high specificity to the protein or partial protein and that is displaced from the binding site by a substance to be tested that likewise binds. Labeling is understood to mean an artificial modification of the molecule to facilitate detection. Examples are radioactive, fluorescent and luminescent labeling.

In another preferred measuring method for determining the change in functional parameters induced in the process according to the invention by the binding of the substance to partial protein or protein, the measurement of at least one of the functional parameters changed by the test substance is carried out via measurement of the regulation, inhibition and/or activation of receptors, ion channels and/or enzymes, especially via measurement of the change in the gene expression, the ionic environment, the pH or the membrane potential, via change in the enzyme activity or the concentration of the 2nd messenger. This covers on one hand the measurement of the action of the substance directly via the influencing of receptors, ion channels and/or enzymes, and, on the other hand, examples of changing parameters that are preferably to be measured, such as gene expression, ionic environment, pH, membrane potential, enzyme activity or concentration of the 2nd messenger. Ionic environment is understood especially to mean the concentration of one or more ions in a cell compartment, especially the cytosol, membrane potential is understood to mean the difference in charge between two sides of a biological membrane, and 2nd messenger is understood to mean messenger substances of the intracellular signal pathway, for example, cyclic AMP (cAMP), inosotol triphosphate (IP3) or diacyl glycerol (DAG).

The invention preferably provides a process according to the invention in which a first process according to the invention is coupled with a second process according to the invention in such a manner that the measured values and results of the first process with regard to the substance to be measured are compared with the measured values and results of the second process with regard to the substance to be measured, characterized in that in one of the two processes, referred to hereinbelow as the main process, in step (a) the substance to be tested is

either

with a biomolecule selected from group II:

the protein BNPI and/or a protein according to one of FIGS. 1b) (SEQ ID NO:2), 1d) (SEQ ID NO:4), 1f) (SEQ ID NO:6) or 1h) (SEQ ID NO:8) and/or a protein that is at least 90% similar to one of these above-mentioned proteins and/or a protein coded for by a polynucleotide according to one of FIGS. 1a) (SEQ ID NO:3), 1c) (SEQ ID NO:3), 1e) (SEQ ID NO:5) or 1g) (SEQ ID NO:7) or by a polynucleotide that is at least 90% similar thereto, and/or a protein coded for by a nucleic acid that binds under stringent conditions to a polynucleotide according to one of FIGS. 1a) (SEQ ID NO:3), 1c) (SEQ ID NO:3), 1e) (SEQ ID NO:5) or 1g) (SEQ ID NO:7) or their antisense polynucleotides, or a partial protein of one of the above-mentioned proteins that has a length of at least 10, preferably at least 15, especially at least 20 amino acids,

and/or a cell and/or a preparation of such a cell that has synthesized at least one of the above-mentioned proteins and partial proteins, or biomolecules of group II, or

with a biomolecule selected from group III:

the protein DNPI and/or a protein according to one of FIGS. 2b) (SEQ ID NO:10), 2d) (SEQ ID NO:12) or 2f) (SEQ ID NO:14) and/or a protein that is at least 90% similar to one of these above-mentioned proteins and/or a protein coded for by a polynucleotide according to one of FIGS. 2a) (SEQ ID NO:9), 2c) (SEQ ID NO:11) or 2e) (SEQ ID NO:13) or by a polynucleotide that is at least 90% similar thereto, and/or a protein coded for by a nucleic acid that binds under stringent conditions to a polynucleotide according to one of FIGS. 2a) (SEQ ID NO:9), 2c) (SEQ ID NO:11) or 2e) (SEQ ID NO:13) or their antisense polynucleotides, or a partial protein of one of the above-mentioned proteins that has a length of at least 10, preferably at least 15, especially at least 20 amino acids, and/or a cell and/or a preparation of such a cell that has synthesized at least one of the above-mentioned proteins and partial proteins, or biomolecules of group III,

and

in that in the other of the two processes, referred to hereinbelow as the subsidiary process, in step (a) the substance to be tested is incubated with a biomolecule from group I or with a biomolecule from that group selected from group II and group III from which the biomolecule with which the substance is incubated in the main process has not been chosen.

This particularly preferred embodiment is to be understood as being especially the combination of on the one hand the measurement of the binding to BNPI or biomolecules derived therefrom or the measurement of the modification of cellular parameters arising therefrom, and on the other hand the binding to DNPI and biomolecules derived therefrom or the measurement of the modification of cellular parameters arising therefrom, because a comparison in view of the totally separate but closely adjacent distribution of the two channels in the tissue can give important information about physiological functions. However, the differential comparison of the data allows the identification of active substances having optimum pharmaceutical or medicinal activity.

Also preferred is a process according to the invention wherein the substances to be found are selected from:

substances having activity in the following indications or for the treatment of visual disturbances, retinitis pigmentosa, optical degeneration, hearing disorders, tinnitus, Menière's disease, hearing loss, amyotrophic lateral sclerosis, neuralgia, weight regulation, obesity, Parkinson's disease, cataracts, viral infections or bacterial infections, diabetic neuropathy, HIV-neuroAIDS; retinal degeneration, glaucoma, nystagmus, detachment of the retina, diseases of the organ of hearing and/or balance, diseases of the auditory canal or vestibular canal, sleep disorders, drug dependency, addiction and withdrawal, especially in the case of alcohol, nicotine, opiates, Ecstasy or cocaine; neuroinflammation, insomnia, for adjuvant therapy by electrostimulation of the nucleus subthalamicus in Parkinson's disease; or substances for the treatment of diseases of the spinal motor neuron, muscular atrophies or muscular dystrophies,

preferably

substances having activity in the following indications or for the treatment of visual disturbances, retinitis pigmentosa, optical degeneration, hearing disorders, tinnitus, Menière's disease, hearing loss, amyotrophic lateral sclerosis, weight regulation, obesity, cataracts, viral infections or bacterial infections, retinal degeneration, glaucoma, nystagmus, detachment of the retina, diseases of the organ of hearing and/or balance, diseases of the auditory canal or vestibular canal, drug dependency, addiction and withdrawal, especially in the case of alcohol, nicotine, opiates, Ecstasy or cocaine; neuroinflammation; or substances for the treatment of diseases of the spinal motor neuron, muscular atrophies or muscular dystrophies,

especially

substances having activity in the following indications or for the treatment of visual disturbances, retinitis pigmentosa, optical degeneration, cataracts, detachment of the retina, retinal degeneration, glaucoma or nystagmus

and/or

hearing disorders, tinnitus, Menière's disease, hearing loss, diseases of the organ of hearing and/or balance, or diseases of the auditory canal or vestibular canal;

or substances for the treatment of diseases of the spinal motor neuron, muscular atrophies or muscular dystrophies.

The invention also provides a compound identifiable by a process according to the invention as a pharmaceutically relevant substance having activity in at least one of the above-mentioned indications. Compound here refers especially to low molecular weight active substances, but also to peptides, proteins and nucleic acids. Identifiable means that the compound has the feature that, in the screening process according to the invention, in respect of binding, it binds markedly more strongly, preferably twice as strongly, as the average of the substances to be tested or, in respect of the change in the functional parameters, it differs markedly from the average of the substances to be tested. It is particularly preferred if the compound according to the invention is a low molecular weight compound.

The invention relates also to the use

a. of a polynucleotide, preferably a DNA or RNA, coding for BNPI or DNPI or of a polynucleotide, preferably a DNA or RNA, which corresponds to the extent of at least 90%, preferably at least 95%, especially at least 97%, or corresponds exactly, to one of the nucleotide sequences shown in one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13),

b. of a polynucleotide, especially an antisense polynucleotide or a PNA, preferably a DNA enzyme or ribozyme, of a ribozyme or other DNA enzyme or of a catalytic RNA or DNA which contains a nucleotide sequence capable of binding specifically to one of the polynucleotides listed under point a),

c. of a vector containing a polynucleotide according to one of points a) or b), especially an expression vector and/or especially a vector derived from a virus, for example the adenovirus, adeno-associated virus or herpes virus, and/or especially a vector containing at least one LTR, poly A, promoter and/or ORI sequence,

d. of BNPI and/or DNPI and/or of a protein according to one of FIGS. 1b) (SEQ ID NO:2), 1d) (SEQ ID NO:4), 1f) (SEQ ID NO:6), 1h) (SEQ ID NO:8), 2b) (SEQ ID NO:10), 2d) (SEQ ID NO:12) or 2f) (SEQ ID NO:14) and/or of a protein that is at least 90%, preferably at least 95%, especially at least 97% similar to one of these above-mentioned proteins, and/or of a protein coded for by a polynucleotide according to one of FIGS. 1a) (SEQ ID NO:l), 1c) (SEQ ID 1NO:3), 1e) (SEQ ID NO:5), 1g) (SEQ ID NO:7), 2a) (SEQ ID NO:9), 2c) (SEQ ID NO:11) or 2e) (SEQ ID NO: 13) or by a polynucleotide that is at least 90%, preferably at least 95%, especially at least 97% similar thereto, and/or of a protein coded for by a nucleic acid that binds under stringent conditions to a polynucleotide according to one of FIGS. 1a) (SEQ ID NO:l), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or its antisense polynucleotides, or of a partial protein of one of the above-mentioned proteins that has a length of at least 10, preferably at least 15, especially at least 20 amino acids, the protein or partial protein optionally having been post-translationally modified, especially glycosylated, phosphorylated, amidated, methylated, acetylated, ADP-ribosylated, hydroxylated, provided with a membrane anchor, cleaved or shortened,

e. of an antibody, preferably a monoclonal or polyclonal antibody, against one of the proteins or partial proteins according to point d),

f. of a cell, preferably an amphibian cell, bacterial cell, yeast cell, insect cell or an immortalized or native mammalian cell, containing a polynucleotide according to one of points a) or b), a vector according to point c), a protein or partial protein according to point d) or an antibody according to point e),

g. of a compound according to the invention identifiable as a pharmaceutically relevant substance having activity in at least one of the above-mentioned indications as described above, and/or

h. of an active substance, preferably a low molecular weight active substance, which binds to a protein or partial protein according to point d),

in the preparation of a medicament for the treatment of visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischemic attacks), emesis, especially hyperemesis, for example during chemotherapy, dizziness in any form, cataracts, arthritis, hyperactivity, developmental disorders, rabies, viral infections or bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, especially glutamate-mediated oversensitivity, neurodegeneration especially in Alzheimer's disease, Alzheimer's disease, ischemia; encephalitis, especially viral or bacterial encephalitis; prion disease, Rasmussen's encephalitis, HIV encephalitis, demyelinisation especially in multiple sclerosis, retinal degeneration, glaucoma, nystagmus, detachment of the retina, diseases of the cerebellum, cerebellar ataxia, diseases of the basal ganglia, diseases of the pallidum, diseases of the organ of hearing and/or balance, diseases of the auditory canal or vestibular canal, memory disorders, learning disorders, cognitive disorders, stiff man syndrome, restless leg syndrome, anxiety, phobias, sleep disorders; drug dependency, addiction and withdrawal, especially in the case of alcohol, nicotine, opiates, Ecstasy or cocaine; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, such as impotence or priapism, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease.

The invention also provides a medicament containing at least

a. a polynucleotide, preferably a DNA or RNA, coding for BNPI or DNPI or a polynucleotide, preferably a DNA or RNA, which corresponds to the extent of at least 90%, preferably at least 95%, especially at least 97%, or corresponds exactly, to one of the nucleotide sequences shown in one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13),

b. a polynucleotide, especially an antisense polynucleotide or a PNA, preferably a DNA enzyme or ribozyme, a ribozyme or other DNA enzyme or a catalytic RNA or DNA which contains a nucleotide sequence capable of binding specifically to one of the polynucleotides listed under point a),

c. a vector containing a polynucleotide according to one of points a) or b), especially an expression vector and/or especially a vector derived from a virus, for example the adenovirus, adeno-associated virus, poly A, promoter and/or ORI sequence,

d. BNPI and/or DNPI and/or a protein according to one of FIGS. 1b) (SEQ ID NO:2), 1d) (SEQ ID NO:4), 1f) (SEQ ID NO:6), 1h) (SEQ ID NO:8), 2b) (SEQ ID NO:10), 2d) (SEQ ID NO:12) or 2f) (SEQ ID NO: 14) and/or a protein that is at least 90%, preferably at least 95%, especially at least 97% similar to one of these above-mentioned proteins, and/or a protein coded for by a polynucleotide according to one of FIGS. 1a) (SEQ ID NO:l), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or by a polynucleotide that is at least 90%, preferably at least 95%, especially at least 97% similar thereto, and/or a protein coded for by a nucleic acid that binds under stringent conditions to a polynucleotide according to one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or its antisense polynucleotides, or a partial protein of one of the above-mentioned proteins that has a length of at least 10, preferably at least 15, especially at least 20 amino acids, the protein or partial protein optionally having been post-translationally modified, especially glycosylated, phosphorylated, amidated, methylated, acetylated, ADP-ribosylated, hydroxylated, provided with a membrane anchor, cleaved or shortened,

e. an antibody, preferably a monoclonal or polyclonal antibody, against one of the proteins or partial proteins according to point d),

f. a cell, preferably an amphibian cell, bacterial cell, yeast cell, insect cell or an immortalized or native mammalian cell, containing a polynucleotide according to one of points a) or b), a vector according to point c), a protein or partial protein according to point d) or an antibody according to point e),

g. a compound according to the invention identifiable as a pharmaceutically relevant substance having activity in at least one of the above-mentioned indications as described above, and/or

h. an active substance, preferably a low molecular weight active substance, which binds to a protein or partial protein according to point d),

and optionally containing suitable additives and/or excipients and/or optionally further active substances.

In addition to at least one compound used in accordance with the invention, the medicaments according to the invention optionally contain suitable additives and/or excipients, for example carriers, fillers, solvents, diluents, colorings and/or binders, and may be administered as liquid forms of administration in the form of injectable solutions, drops or juices, as semi-solid forms of administration in the form of granules, tablets, pellets, patches, capsules, plasters or aerosols. The choice of excipients etc. and the amounts thereof to be used depend on whether the medicament is to be administered orally, perorally, parenterally, intravenously, intraperitoneally, intradermally, intramuscularly, intranasally, buccally, rectally or locally, for example to the skin, the mucous membranes or into the eyes. There are suitable for oral administration preparations in the form of tablets, dragées, capsules, granules, drops, juices and syrups, and for parenteral and topical administration and for administration by inhalation there are suitable solutions, suspensions, readily reconstitutable dry preparations and also sprays. Compounds used in accordance with the invention in depot form, in dissolved form or in a plaster, optionally with the addition of agents promoting penetration of the skin, are suitable percutaneous forms of administration. Forms of preparation for oral or percutaneous administration may release the compounds used in accordance with the invention in a delayed manner. In principle, other further active substances known to the person skilled in the art can be added to the medicaments according to the invention.

The amount of active substance to be administered to the patient varies in dependence on the weight of the patient, the mode of administration, the indication and the degree of severity of the disease. From 0.005 to 1000 mg/kg are usually administered.

The invention relates also to the use

a. of a polynucleotide, preferably a DNA or RNA, coding for BNPI or DNPI or of a polynucleotide, preferably a DNA or RNA, which corresponds to the extent of at least 90%, preferably at least 95%, especially at least 97%, to one of the nucleotide sequences shown in one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13),

b. of a polynucleotide, especially an antisense polynucleotide or a PNA, preferably a DNA enzyme or ribozyme, of a ribozyme or other DNA enzyme or of a catalytic RNA or DNA which contains a nucleotide sequence capable of binding specifically to one of the polynucleotides listed under point a),

c. of a vector containing a polynucleotide according to one of points a) or b), especially an expression vector and/or especially a vector derived from a virus, for example the adenovirus, adeno-associated virus or herpes virus, and/or especially a vector containing at least one LTR, poly A, promoter and/or ORI sequence,

f. of a cell, preferably an amphibian cell, bacterial cell, yeast cell, insect cell or an immortalized or native mammalian cell, containing a polynucleotide according to one of points a) or b) or a vector according to point c),

in the preparation of a medicament for use in gene therapy. It is particularly preferred for the gene therapy to be in vivo or in vitro gene therapy. Gene therapy is understood to be a form of therapy in which an effector gene, in most cases a protein, is expressed by the introduction of nucleic acids into cells. A distinction is made in principle between in vivo and in vitro processes. In in vitro processes, cells are removed from the organism and transfected with vectors ex vivo in order subsequently to be introduced into the same organism again or into a different organism. In in vivo gene therapy, vectors, for example for controlling tumors, are administered systemically (e.g., via the bloodstream) or directly into the target tissue (e.g., into a tumor).

In the case of use in gene therapy it is further preferred for the medicament to be a medicament having activity in the following indications or for the treatment of visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischemic attacks), emesis, especially hyperemesis, for example during chemotherapy, dizziness in any form, cataracts, arthritis, hyperactivity, developmental disorders, rabies, viral infections or bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, especially glutamate-mediated oversensitivity, neurodegeneration especially in Alzheimer's disease, Alzheimer's disease, ischemia; encephalitis, especially viral or bacterial encephalitis; prion disease, Rasmussen's encephalitis, HIV encephalitis, demyelinisation especially in multiple sclerosis, retinal degeneration, glaucoma, nystagmus, detachment of the retina, diseases of the cerebellum, cerebellar ataxia, diseases of the basal ganglia, diseases of the pallidum, diseases of the organ of hearing and/or balance, diseases of the auditory canal or vestibular canal, memory disorders, learning disorders, cognitive disorders, stiff man syndrome, restless leg syndrome, anxiety, phobias, sleep disorders; drug dependency, addiction and withdrawal, especially in the case of alcohol, nicotine, opiates, Ecstasy or cocaine; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, such as impotence or priapism, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease.

In the case of use in gene therapy, preference is given also to the use of a polynucleotide which is an antisense polynucleotide or PNA, or which is part of a ribozyme or other DNA enzyme or of a catalytic RNA or DNA.

The invention relates also to the use

a. of a polynucleotide, preferably a DNA or RNA, coding for BNPI or DNPI or of a polynucleotide, preferably a DNA or RNA, which corresponds to the extent of at least 90%, preferably 95%, especially at least 97%, to one of the nucleotide sequences shown in one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13),

b. of a polynucleotide, especially an antisense polynucleotide or a PNA, preferably a DNA enzyme or ribozyme, of a ribozyme or other DNA enzyme or of a catalytic RNA or DNA which contains a nucleotide sequence capable of binding specifically to one of the polynucleotides listed under point a),

c. of a vector containing a polynucleotide according to one of points a) or b), especially an expression vector and/or especially a vector derived from a virus, for example the adenovirus, adeno-associated virus or herpes virus, and/or especially a vector containing at least one LTR, poly A, promoter and/or ORI sequence,

d. of BNPI and/or DNPI and/or of a protein according to one of FIGS. 1b) (SEQ ID NO:2), 1d) (SEQ ID NO:4), 1f) (SEQ ID NO:6), 1h) (SEQ ID NO:8), 2b) (SEQ ID NO:10), 2d) (SEQ ID NO:12) or 2f) (SEQ ID NO:14) and/or of a protein that is at least 90%, preferably at least 95%, especially at least 97% similar to one of these above-mentioned proteins, and/or of a protein coded for by a polynucleotide according to one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or by a polynucleotide that is at least 90%, preferably at least 95%, especially at least 97% similar thereto, and/or of a protein coded for by a nucleic acid that binds under stringent conditions to a polynucleotide according to one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or its antisense polynucleotides, or of a partial protein of one of the above-mentioned proteins that has a length of at least 10, preferably at least 15, especially at least 20 amino acids, the protein or partial protein optionally having been post-translationally modified, especially glycosylated, phosphorylated, amidated, methylated, acetylated, ADP-ribosylated, hydroxylated, provided with a membrane anchor, cleaved or shortened,

e. of an antibody, preferably a monoclonal or polyclonal antibody, against one of the proteins or partial proteins according to point d),

f. of a cell, preferably an amphibian cell, bacterial cell, yeast cell, insect cell or an immortalized or native mammalian cell, containing a polynucleotide according to one of points a) or b), a vector according to point c), a protein or partial protein according to point d) or an antibody according to point e),

g. of a compound according to the invention identifiable as a pharmaceutically relevant substance having activity in at least one of the above-mentioned indications as described above, and/or

h. of an active substance, preferably a low molecular weight active substance, which binds to a protein or partial protein according to point d),

in treatment methods for, and the preparation of a diagnostic agent for the diagnosis of, a condition selected from visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischemic attacks), emesis, especially hyperemesis, for example during chemotherapy, dizziness in any form, cataracts, arthritis, hyperactivity, developmental disorders, rabies, viral infections or bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, especially glutamate-mediated oversensitivity, neurodegeneration especially in Alzheimer's disease, Alzheimer's disease, ischemia; encephalitis, especially viral or bacterial encephalitis; prion disease, Rasmussen's encephalitis, HIV encephalitis, demyelinisation especially in multiple sclerosis, retinal degeneration, glaucoma, nystagmus, detachment of the retina, diseases of the cerebellum, cerebellar ataxia, diseases of the basal ganglia, diseases of the pallidum, diseases of the organ of hearing and/or balance, diseases of the auditory canal or vestibular canal, memory disorders, learning disorders, cognitive disorders, stiff man syndrome, restless leg syndrome, anxiety, phobias, sleep disorders; drug dependency, addiction and withdrawal, especially in the case of alcohol, nicotine, opiates, Ecstasy or cocaine; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, such as impotence or priapism.

Diagnosis is understood to mean the analysis of symptoms associated with a disease pattern, and activity studies are understood to mean studies of the activity of substances to be tested, especially their medicinal activity.

The invention also provides a process for the preparation of a peptide or protein according to the invention, in which a cell according to the invention that contains a polynucleotide according to the invention and/or a vector according to the invention is cultivated and the peptide or protein is optionally isolated.

The invention relates also to the use

a. of a polynucleotide, preferably a DNA or RNA, coding for BNPI or DNPI or of a polynucleotide, preferably a DNA or RNA, which corresponds to the extent of at least 90%, preferably 95%, especially at least 97%, to one of the nucleotide sequences shown in one of FIGS. 1a) (SEQ ID NO:l), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13),

b. of a polynucleotide, especially an antisense polynucleotide or a PNA, preferably a DNA enzyme or ribozyme, of a ribozyme or other DNA enzyme or of a catalytic RNA or DNA which contains a nucleotide sequence capable of binding specifically to one of the polynucleotides listed under point a),

c. of a vector containing a polynucleotide according to one of points a) or b), especially an expression vector and/or especially a vector derived from a virus, for example the adenovirus, adeno-associated virus or herpes virus, and/or especially a vector containing at least one LTR, poly A, promoter and/or ORI sequence,

d. of BNPI and/or DNPI and/or of a protein according to one of FIGS. 1b) (SEQ ID NO:2), 1d) (SEQ ID NO:4), 1f) (SEQ ID NO:6), 1h) (SEQ ID NO:8), 2b) (SEQ ID NO:10), 2d) (SEQ ID NO:12) or 2f) (SEQ ID NO:14) and/or of a protein that is at least 90%, preferably at least 95%, especially at least 97% similar to one of these above-mentioned proteins, and/or of a protein coded for by a polynucleotide according to one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or by a polynucleotide that is at least 90%, preferably at least 95%, especially at least 97% similar thereto, and/or of a protein coded for by a nucleic acid that binds under stringent conditions to a polynucleotide according to one of FIGS. 1a) (SEQ ID NO:1), 1c) (SEQ ID NO:3), 1e) (SEQ ID NO:5), 1g) (SEQ ID NO:7), 2a) (SEQ ID NO:9), 2c) (SEQ ID NO:11) or 2e) (SEQ ID NO:13) or its antisense polynucleotides, or of a partial protein of one of the above-mentioned proteins that has a length of at least 10, preferably at least 15, especially at least 20 amino acids, the protein or partial protein optionally having been post-translationally modified, especially glycosylated, phosphorylated, amidated, methylated, acetylated, ADP-ribosylated, hydroxylated, provided with a membrane anchor, cleaved or shortened,

e. of an antibody, preferably a monoclonal or polyclonal antibody, against one of the proteins or partial proteins according to point d),

f. of a cell, preferably an amphibian cell, bacterial cell, yeast cell, insect cell or an immortalized or native mammalian cell, containing a polynucleotide according to one of points a) or b), a vector according to point c), a protein or partial protein according to point d) or an antibody according to point e), in a process for finding pharmaceutically relevant substances having activity in the following indications or for the treatment of visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischemic attacks), emesis, especially hyperemesis, for example during chemotherapy, dizziness in any form, cataracts, arthritis, hyperactivity, developmental disorders, rabies, viral infections or bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, especially glutamate-mediated oversensitivity, neurodegeneration especially in Alzheimer's disease, Alzheimer's disease, ischemia; encephalitis, especially viral or bacterial encephalitis; prion disease, Rasmussen's encephalitis, HIV encephalitis, demyelinisation especially in multiple sclerosis, retinal degeneration, glaucoma, nystagmus, detachment of the retina, diseases of the cerebellum, cerebellar ataxia, diseases of the basal ganglia, diseases of the pallidum, diseases of the organ of hearing and/or balance, diseases of the auditory canal or vestibular canal, memory disorders, learning disorders, cognitive disorders, stiff man syndrome, restless leg syndrome, anxiety, phobias, sleep disorders; drug dependency, addiction and withdrawal, especially in the case of alcohol, nicotine, opiates, Ecstasy or cocaine; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, such as impotence or priapism, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease.

In general, it is preferred in all the above-mentioned uses according to the invention for the indication or the disease to be treated or diagnosed to be selected from

visual disturbances, retinitis pigmentosa, optical degeneration, hearing disorders, tinnitus, Menière's disease, hearing loss, amyotrophic lateral sclerosis, neuralgia, weight regulation, obesity, Parkinson's disease, cataracts, viral infections or bacterial infections, diabetic neuropathy, autoimmune diabetes, alcoholic neuropathy, HIV-neuroAIDS; retinal degeneration, glaucoma, nystagmus, detachment of the retina, diseases of the organ of hearing and/or balance, diseases of the auditory canal or vestibular canal, sleep disorders; drug dependency, addiction and withdrawal, especially in the case of alcohol, nicotine, opiates, Ecstasy or cocaine; neuroinflammation, insomnia, for adjuvant therapy by electrostimulation of the nucleus subthalamicus in Parkinson's disease; or diseases of the spinal motor neuron, muscular atrophies or muscular dystrophies,

preferably

visual disturbances, retinitis pigmentosa, optical degeneration, hearing disorders, tinnitus, Menière's disease, hearing loss, amyotrophic lateral sclerosis, weight regulation, obesity, cataracts, viral infections or bacterial infections, retinal degeneration, glaucoma, nystagmus, detachment of the retina, diseases of the organ of hearing and/or balance, diseases of the auditory canal or vestibular canal, drug dependency, addiction and withdrawal, especially in the case of alcohol, nicotine, opiates, Ecstasy or cocaine; neuroinflammation; or diseases of the spinal motor neuron, muscular atrophies or muscular dystrophies,

especially

visual disturbances, retinitis pigmentosa, optical degeneration, cataracts, detachment of the retina, retinal degeneration, glaucoma or nystagmus and/or

hearing disorders, tinnitus, Menière's disease, hearing loss, diseases of the organ of hearing and/or balance, or diseases of the auditory canal or vestibular canal;

or diseases of the spinal motor neuron, muscular atrophies or muscular dystrophies.

The polynucleotide used in accordance with the invention also includes the illustrated gene fragments themselves as well as a polynucleotide that corresponds either fully or at least to parts of the coding sequence of the gene corresponding to the fragment. This also includes polynucleotides whose base sequence corresponds to at least 90%, preferably 95%, especially at least 97%, of the coding sequence of the illustrated polynucleotides or the coding sequence of the gene. It is further preferred for the polynucleotide to be RNA or single- or double-stranded DNA, especially mRNA or cDNA. It is likewise preferred for the polynucleotide to be an antisense polynucleotide or PNA which contains a sequence capable of binding specifically to a polynucleotide according to the invention. PNA is understood to mean peptidic nucleic acid, which carries the base pairs but whose backbone is peptidically bonded. An antisense polynucleotide exhibits the complementary base sequence to at least a portion of a base nucleic acid. It is likewise preferred for the polynucleotide to be part of a ribozyme or other DNA enzyme or of a catalytic RNA or DNA. Ribozyme is understood to mean a catalytically active ribonucleic acid; DNA enzyme is understood to mean a corresponding deoxyribonucleic acid, that is to say catalytic RNA or DNA.

The invention very particularly preferably provides a polynucleotide, especially a polynucleotide used in accordance with the invention, or an oligonucleotide in which at least one of the nucleotides, especially more than one nucleotide, are locked nucleic acids (LNA's) or at least one of the nucleotides, especially all the nucleotides, are phosphorothiates, preferably one in which more than one of the nucleotides are locked nucleic acids (LNA's). Locked nucleic acids (LNA's) are ribonucleotides which contain a methylene bridge which binds the 2′-oxygen of the ribose to the 4′-oxygen (see FIG. 27). An overview of LNA's is given by Braasch D. A. and Corey, D. R. (2001), Locked nucleic acids (LNA); fine-tuning the recognition of DNA and RNA. Chem. Biol. 8, 1-7. This article is incorporated by reference in the present description and disclosure. LNA's are supplied, for example, by Proligo, Boulder, Colo., USA. Phosphorothiates are also known to the person skilled in the art and can be ordered, for example, from MWG-Biotech AG, Ebersberg, Germany.

The vector used in accordance with the invention is understood to be a nucleic acid molecule which is used in manipulation by genetic engineering to contain or to transfer foreign genes. It is particularly preferably an expression vector. It serves to express the foreign gene, the polynucleotide, it contains. Further preferred is such a vector derived from a virus, for example the adenovirus, adeno-associated virus or herpes virus, and/or it contains at least one LTR, poly A, promoter and/or ORI sequence. A LTR is a long-terminal repeat, a section located at the end, for example in viruses. A poly-A sequence is a tail more than 20 adenosine residues long. A promoter sequence is the control region for transcription.

A protein that is used, or a partial protein derived therefrom, has preferably been post-translationally modified, especially glycosylated, phosphorylated, amidated, methylated, acetylated, ADP-ribosylated, hydroxylated, provided with a membrane anchor, cleaved or shortened. Post-translational modifications are to be found, for example, in Voet/Voet, Biochemistry, 1st Edition, 1990, p. 935-938.

It is particularly preferred for a use according to the invention for the polynucleotide (optionally according to point a) and/or point b)) to be a RNA or a single- or double-stranded DNA, especially mRNA or cDNA.

It is particularly preferred for a use according to the invention for the polynucleotide (optionally according to point b)) to be part of a ribozyme or other DNA enzyme or of a catalytic RNA or DNA.

It is particularly preferred for a use according to the invention for the vector (optionally according to point c)) to be an expression vector.

It is further particularly preferred for a use according to the invention for the vector (optionally according to point c)) to be derived from a virus, for example the adenovirus, adeno-associated virus or herpes virus, and/or it contains at least one LTR, poly A, promoter and/or ORI sequence.

It is particularly preferred for a use according to the invention (not gene therapy) for the protein or partial protein (optionally according to point d)) to have been post-translationally modified, especially glycosylated, phosphorylated, amidated, methylated, acetylated, ADP-ribosylated, hydroxylated, provided with a membrane anchor, cleaved or shortened.

It is particularly preferred for a use according to the invention (not gene therapy) for the antibody (optionally according to point e)) to be a monoclonal or polyclonal antibody.

It is particularly preferred for a use according to the invention for the cell (optionally according to point f)) to be an amphibian cell, bacterial cell, yeast cell, insect cell or an immortalized or native mammalian cell.

It is particularly preferred for a use according to the invention for the compound (optionally according to point g)) to be a low molecular weight compound.

It is particularly preferred for a use according to the invention for the mentioned active substance according to point h) to be a low molecular weight active substance.

The invention also provides a method of treating a non-human mammal or a human being requiring treatment of visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, Alzheimer's disease, TIA (transient ischemic attacks), emesis, especially hyperemesis, for example during chemotherapy, dizziness in any form, cataracts, arthritis, hyperactivity, developmental disorders, rabies, viral infections or bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, especially glutamate-mediated oversensitivity, neurodegeneration especially in Alzheimer's disease, Alzheimer's disease, ischemia; encephalitis, especially viral or bacterial encephalitis; prion disease, Rasmussen's encephalitis, HIV encephalitis, amyotrophic lateral sclerosis, demyelinisation especially in multiple sclerosis, retinal degeneration, glaucoma, nystagmus, detachment of the retina, diseases of the cerebellum, cerebellar ataxia, diseases of the basal ganglia, diseases of the pallidum, diseases of the organ of hearing and/or balance, diseases of the auditory canal or vestibular canal, memory disorders, learning disorders, cognitive disorders, stiff man syndrome, restless leg syndrome, anxiety, phobias, sleep disorders, anorexia nervosa; drug dependency, addiction and withdrawal, especially in the case of alcohol, nicotine, opiates, Ecstasy or cocaine; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, such as impotence or priapism, or promotion of microglia activation, of learning, of cognition or of memory, neuroprotection, liquor diagnosis of neurostatic diseases or adjuvant therapy by electrostimulation of the nucleus subthalamicus in Parkinson's disease, by administration of a medicament according to the invention, especially such a medicament containing a substance according to the invention and/or an active substance that binds to BNPI and/or DNPI.

The administration may be carried out, for example, in the form of a medicament as described above.

Certain embodiments of the present invention may be understood more readily by reference to the figures and specific examples. The following examples, figures and the terminology used herein are for the purpose of describing particular embodiments and are intended to illustrate the invention without limiting it thereto.

### FIGURES AND EXAMPLES

Figures:

FIG. 1a) cDNA sequence of BNPI, human; AN: NM_{—}020309

FIG. 1b) Amino acid sequence of BNPI, human; AN: NM_{—}020309

FIG. 1c) cDNA sequence of BNPI, human; No.: AAT42064 from WO 96/34288

FIG. 1d) Amino acid sequence of BNPI, human; No.: AAT42064 from WO 96/34288

FIG. 1e) cDNA sequence of BNPI, rat; AN: U07609

FIG. 1f) Amino acid sequence of BNPI, rat; AN: U07609

FIG. 1g) cDNA sequence of BNPI, mouse; AN: XM_{—}133432

FIG. 1h) Amino acid sequence of BNPI, mouse: AN: XM_{—}133432

FIG. 2a) cDNA sequence of DNPI, human; AN: AB032435

FIG. 2b) Amino acid sequence of DNPI, human; AN: AB032435

FIG. 2c) cDNA sequence of DNPI, rat; AN: AF271235

FIG. 2d) Amino acid sequence of DNPI, rat; AN: AF271235

FIG. 2e) cDNA sequence of DNPI, mouse; AN: NM_{—}080853

FIG. 2f) Amino acid sequence of DNPI, mouse: AN: NM_{—}080853

FIG. 3) Differential expression of DNPI and BNPI in synapses and motor areas of the lumbar spinal cord of the rat (see Example 2a (SEQ ID NO:9)

FIG. 4) Differential expression of DNPI and BNPI in synapses of the dorsal horn areas of the lumbar spinal cord of the rat (see Example 2b) (SEQ ID NO:10)

FIG. 5) Differential expression of DNPI and BNPI in synapses of the sacral spinal cord of the rat (see Example 2c (SEQ ID NO:11))

FIG. 6) Differential expression of DNPI and BNPI in synapses of the medullo-cervicospinal line of the trigeminal nerve of the rat (see Example 2d (SEQ ID NO:12))

FIG. 7) Differential expression of DNPI and BNPI in lower regions of the rat (see Example 2e (SEQ ID NO:13))

FIG. 8) Differential expression of DNPI and BNPI in lower regions of the rat (see Example 2f (SEQ ID NO: 14))

FIG. 9) Differential expression of DNPI and BNPI in lower regions of the rat (see Example 2g (SEQ ID NO:15))

FIG. 10) Differential expression of DNPI and BNPI in lower regions of the rat (see Example 2h (SEQ ID NO:16))

FIG. 11) Differential expression of DNPI and BNPI in lower regions of the rat (see Example 2i (SEQ ID NO:17))

FIG. 12) Differential expression of DNPI and BNPI in lower regions of the rat (see Example 2j (SEQ ID NO:18))

FIG. 13) Differential expression of DNPI and BNPI in lower regions of the rat (see Example 2k (SEQ ID NO:19))

FIG. 14) Differential expression of DNPI and BNPI in lower regions of the rat (see Example 21 (SEQ ID NO:20))

FIG. 15) Differential expression of DNPI and BNPI in lower regions of the rat (see Example 2m (SEQ ID NO:21))

FIG. 16) Differential expression of DNPI and BNPI in lower regions of the rat (see Example 2n (SEQ ID NO:22))

FIG. 17) Differential expression of DNPI and BNPI in lower regions of the rat (see Example 2o (SEQ ID NO:23))

FIG. 18) Differential expression of DNPI and BNPI in lower regions of the rat (see Example 2p (SEQ ID NO:24))

FIG. 19) Differential expression of DNPI and BNPI in lower regions of the rat (see Example 2q (SEQ ID NO:25))

FIG. 20) Differential expression of DNPI and BNPI in lower regions of the rat, where AS means antisense and relates to the staining.

FIG. 21) Differential expression of DNPI and BNPI in lower regions of the rat, where AS means antisense and relates to the staining.

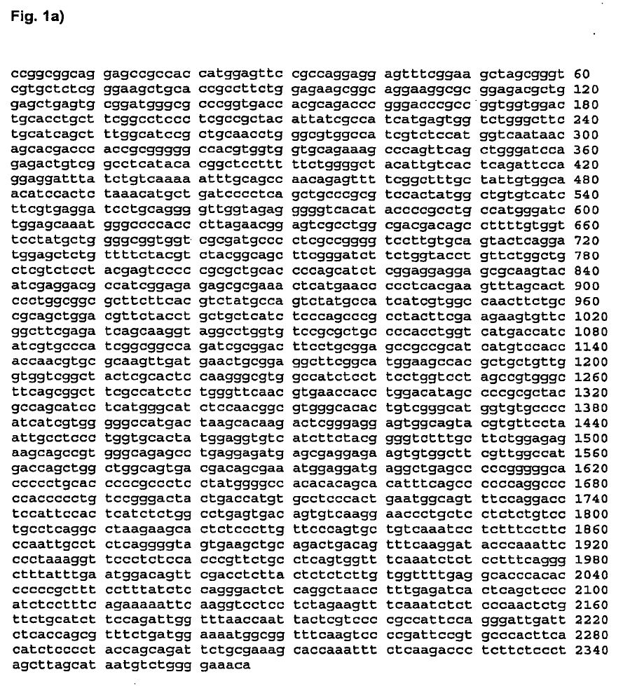

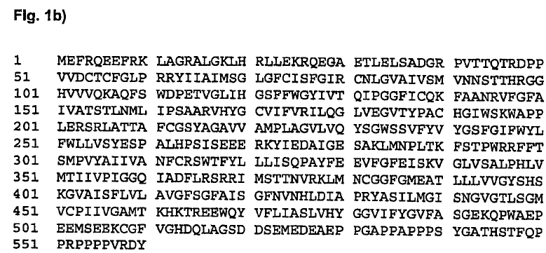

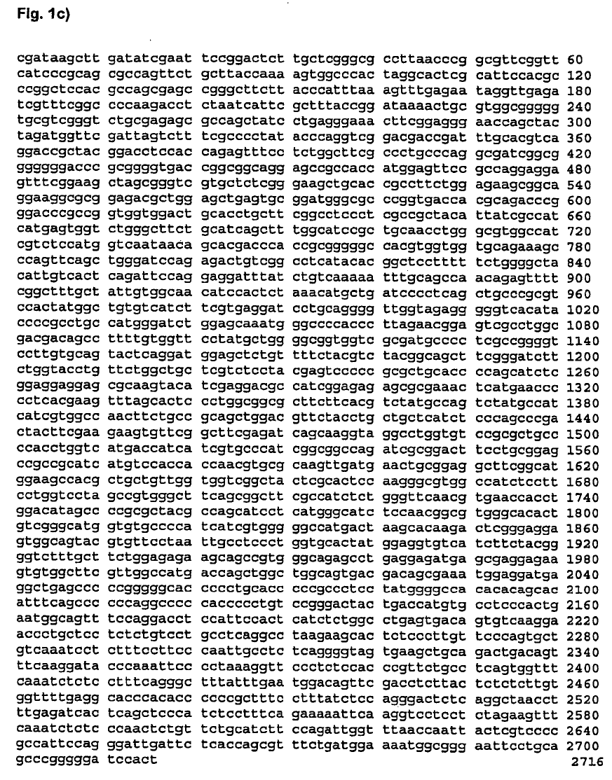

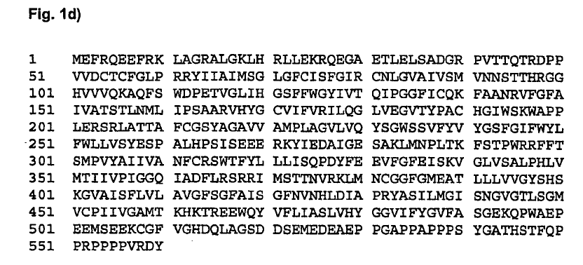

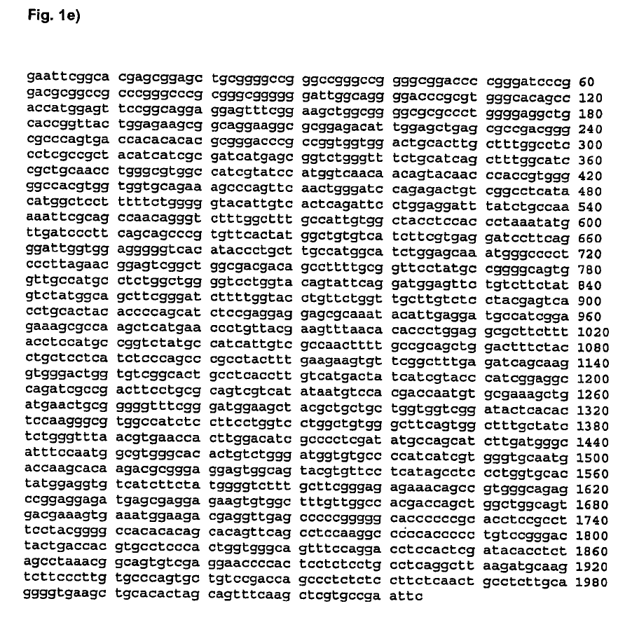

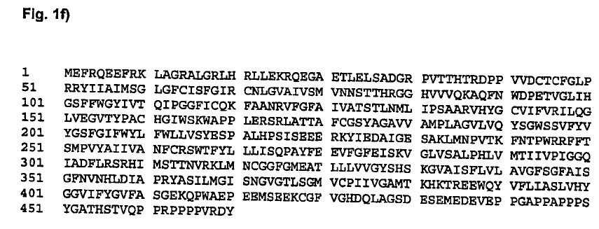

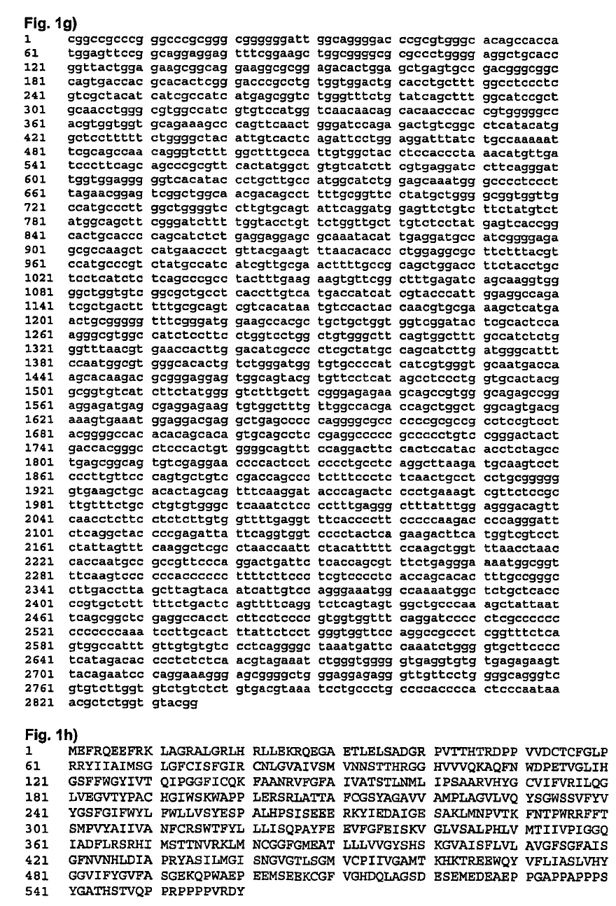

### EXAMPLES

### Example 1

### Differential Consideration of Expression Between DNPI and BNPI Via Immunocytochemical Staining

Polyclonal rabbit antiserums against the recombinant DNPI or BNPI fusion protein were used for the immunohistochemical staining. In general, sections of different regions of the CNS were prepared and the expression of DNPI was compared with that of BNPI. In respect of the sections and staining, the procedure corresponded to the process described by Persson S., Schäfer M K-H., Nohr D., Ekström G., Post C., Nyberg F. and Weihe E. (1994), Neuroscience 63: 313-326 or Nohr D., Schäfer M K-H., Romeo H., Persson S., Nyberg F., Post C. and Weihe E. (1999), Neuroscience 93; 759-773, the disclosure, description and technical teaching of these articles are incorporated by reference into this disclosure, description and technical teachings of this invention.

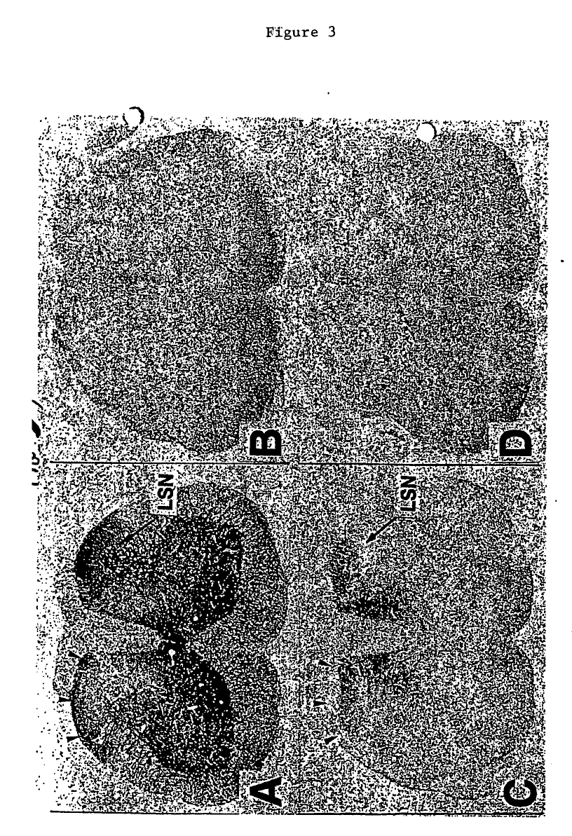

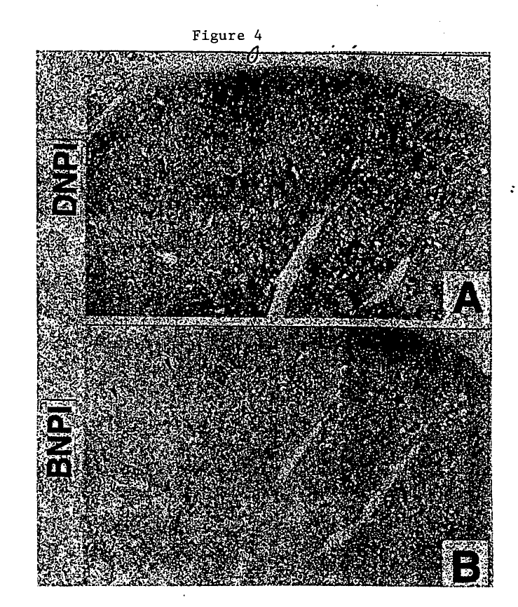

### Example 2a Relating to FIG. 3)

The differential distribution of the immune reactivity of BNPI and DNPI in the lumbar spinal cord of the rat is shown. The adjacent deparaffined sections A to D are stained as follows:

A=anti-DNPI;

B=anti-DNPI pre-adsorbed with DNPI fusion protein;

C=anti-BNPI;

D=anti-BNPI pre-adsorbed with BNPI fusion protein;

The DNPI (A) and BNPI (C) immune stains were wholly pre-adsorbable with homologous recombinant BNPI (D) and BNPI (B) fusion protein, which demonstrates the specificity of the immune reaction.

The mutually exclusive distribution pattern of DNPI and BNPI immune staining in the outer and deep dorsal horn is to be noted. (A;C). Point-like immune staining of DNPI is in the synaptic ends of the outer dorsal horn (lamina 1 and substantia gelatinosa) (arrow in A), whereas BNPI immune reactivity is completely absent (arrows in B). There is accumulation of pronounced positive point-like BNPI immune staining in the deeper dorsal horn, whereas DNPI staining is relatively low. DNPI is present in the lateral spinal nucleus (LSN in A), whereas BNPI is completely absent (LSN in C). DNPI is abundant in the lamina X around the central canal, whereas BNPI is rare. BNPI immune staining is weak in the lateral ventral horn and slight or absent in the medial ventral horn. Point-like DNPI staining is abundant throughout the entire ventral horn, slightly less in the lateral horn compared with the medial ventral horn. There is a weak BNPI and DNPI staining in some cell bodies of the motor neurons located in the ventral horn, which was not, however, pre-adsorbed by the homologous transporter fusion proteins and has therefore been classified as non-specific.

### Example 2b Relating to FIG. 4)

The differential distribution of the immune reactivity of BNPI and DNPI in the left lateral superficial dorsal lumbar spinal cord of the rat is shown. A and B, stained for BNPI (A) and DNPI (B), show many point-like stainings for DNPI, which are concentrated in the lamina I and substantia gelatinosa, where BNPI is almost completely absent. Furthermore, dense complexes of DNPI-positive points are shown in the lateral spinal nucleus, where BNPI is almost completely absent. Fine DNPI-positive points are also found in the deeper dorsal horn, although with a lesser density.

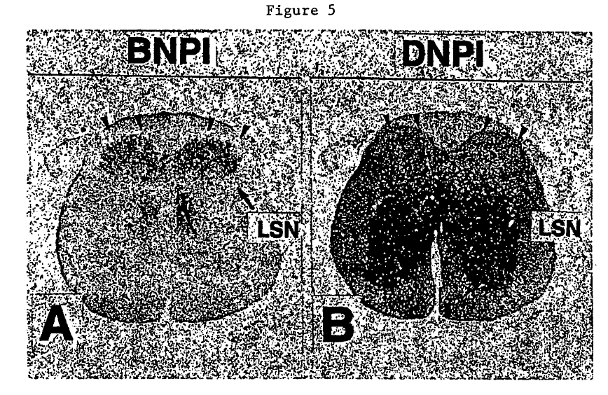

### Example 2c Relating to FIG. 5)

The differential distribution of the immune reactivity of BNPI and DNPI in the sacral spinal cord of the rat is shown. The adjacent sections A and B, stained for BNPI (A) and DNPI (B), show mutual exclusion zones of point-like DNPI and BNPI immune staining in the dorsal horn. DNPI is present in the entire grey matter and is concentrated in the outermost layers of the dorsal horn, where it forms a narrow band at the boundary to white matter. DNPI is abundant in the lateral spinal nucleus and in the lamina X as well as in the lamina V/VI and in the entire ventral horn. BNPI is abundant in the deep dorsal horn and is scarce in the ventral horn.

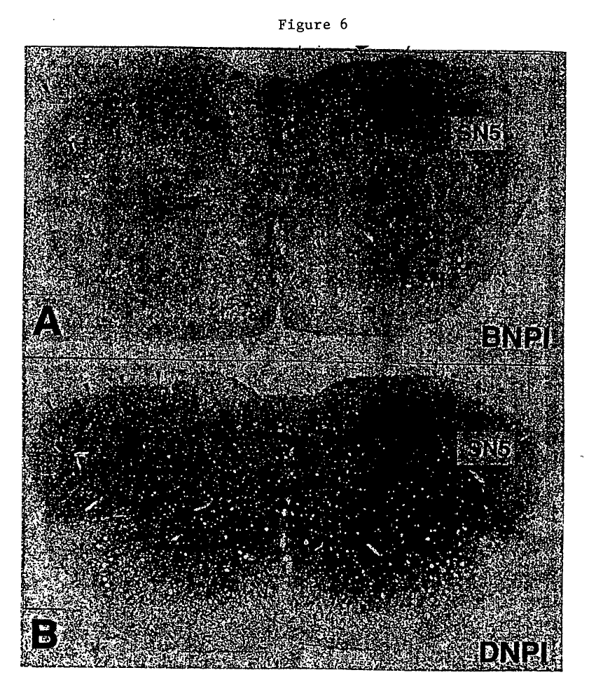

### Example 2d Relating to FIG. 6)

The differential distribution of the immune reactivity of BNPI and DNPI in the lower medulla oblongata at the transition to the cervical spinal cord is shown. The adjacent sections A and B, stained for BNPI (A) and DNPI (B), show a preferential accumulation of BNPI staining in the medial part of the spinal trigeminal nucleus and in the middle and lower part of the dorsal medulla. Only very weak staining with BNPI is shown in the ventral medulla. DNPI is abundant in grey matter of the medulla. DNPI staining overlaps with BNPI staining in the inner spinal nucleus V. It is to be noted that BNPI is also found in the upper spinal trigeminal nucleus, which is the same as the spinal substantia gelatinosa. DNPI staining is weaker in areas in which BNPI is present, weaker than in areas in which BNPI is low or is absent. A small number of BNPI points are shown in the ventral grey motor region.

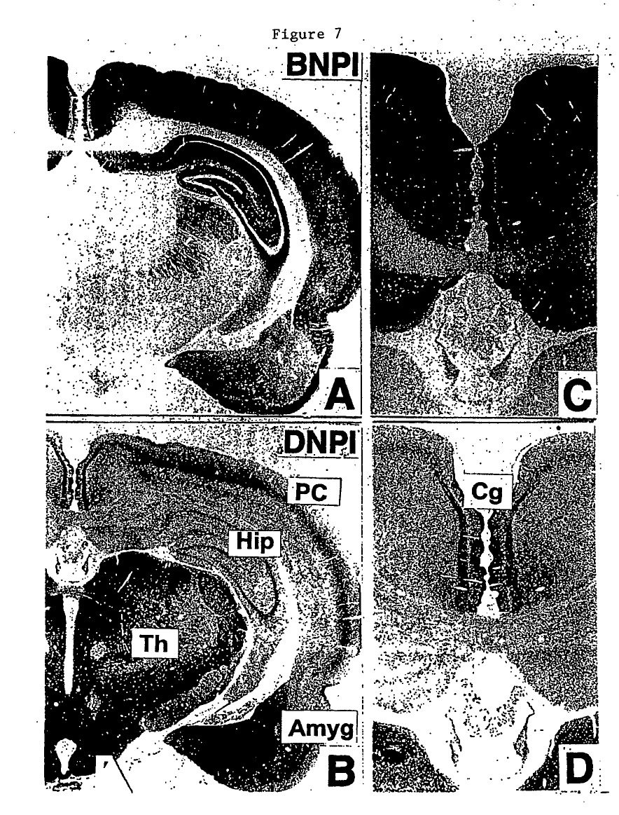

### Example 2e Relating to FIG. 7)

Complementary differential distribution of DNPI and BNPI immune reactivity in 2 successive sections of the rat brain in regions of the brain that are relevant for pain, such as sensory parietal cortex, cingular cortex, thalamus, corpus amygdaloideum and hypothalamus. DNPI is concentrated in the cortex in the granular sensory layers, especially in lamina IV; BNPI is abundant in the cortex but weaker in lamina IV than in other lamina. In the cingular cortex (C vs D as high magnification), the distribution of DNPI and BNPI is complementarily mutually exclusive or reciprocal in the density of the particular synapses. DNPI is clearly predominant over BNPI in the thalamus; BNPI is sparse in the hypothalamus, DNPI is abundant. Abundant BNPI is predominant over sparse DNPI in the hippocampus with mutually complementary distribution.

thalamus=Th,

amygdala=Amyg,

hippocampus=Hip,

cingular cortex=Cg,

hypothalamus=Hy,

parietal cortex=PC.

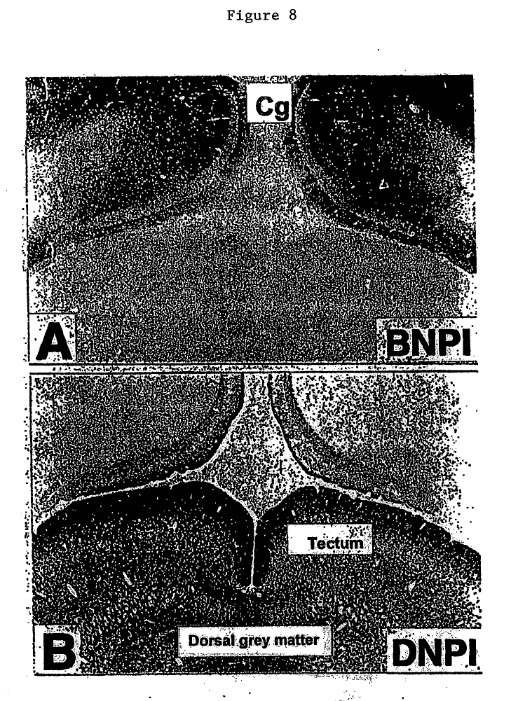

### Example 2f Relating to FIG. 8)

Complementarily differential distribution of DNPI and BNPI immune reactivity in regions of the brain that are relevant for pain, such as the cingular cortex (Cg) and the tectum and the dorsal periaquaductal grey matter. DNPI is dominant in the tectum and dorsal grey matter. Successive sections of a rat brain through the upper mesencephalon.

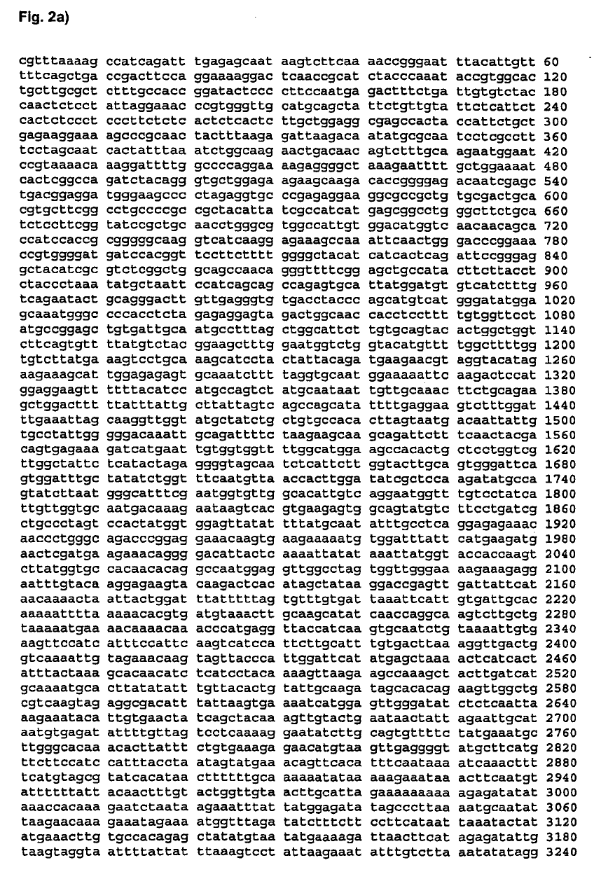

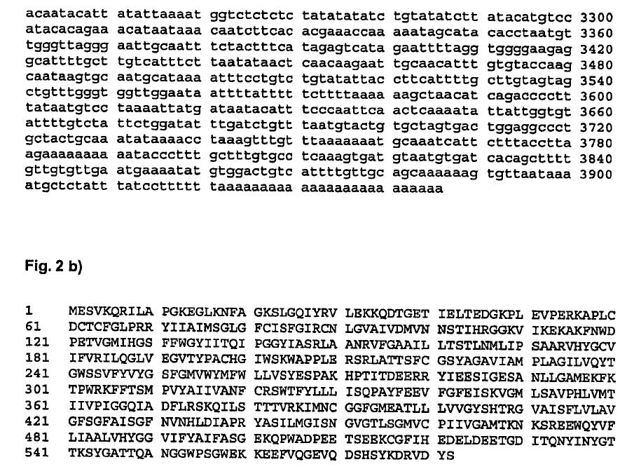

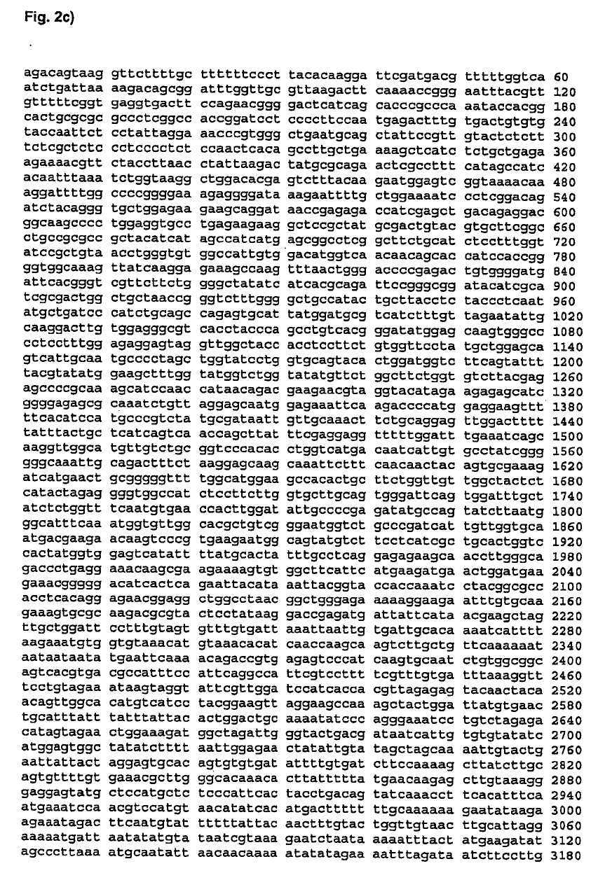

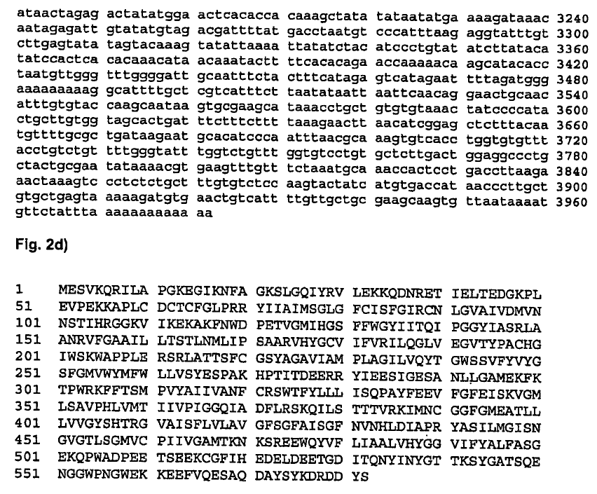

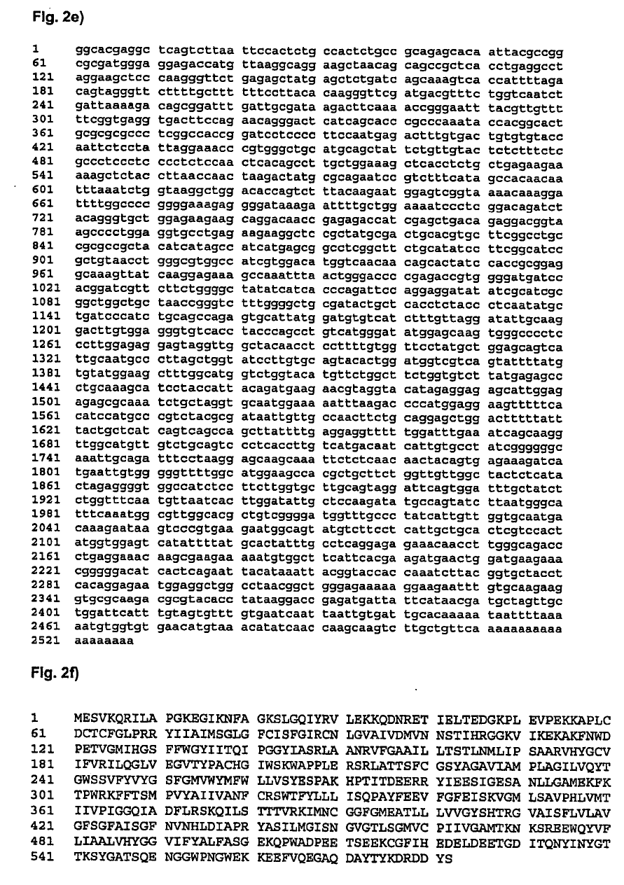

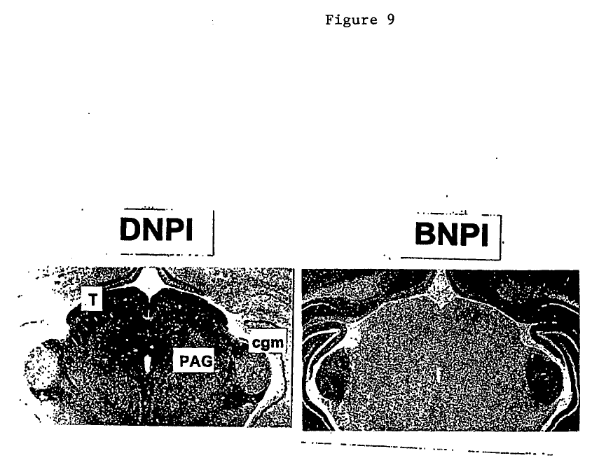

### FIG. 2g Relating to FIG. 9)

Complementarily differential distribution of DNPI and BNPI immune reactivity in regions of the brain that are relevant for pain, such as the tectum (T) and periaquaductal grey matter (PAG). DNPI is dominant in the tectum and dorsal grey matter. Differential distribution of DNPI and BNPI in the corpus geniculatum mediale (cgm) of the auditory canal are noted. Successive sections of a rat brain through the upper mesencephalon; plane colliculus superior.

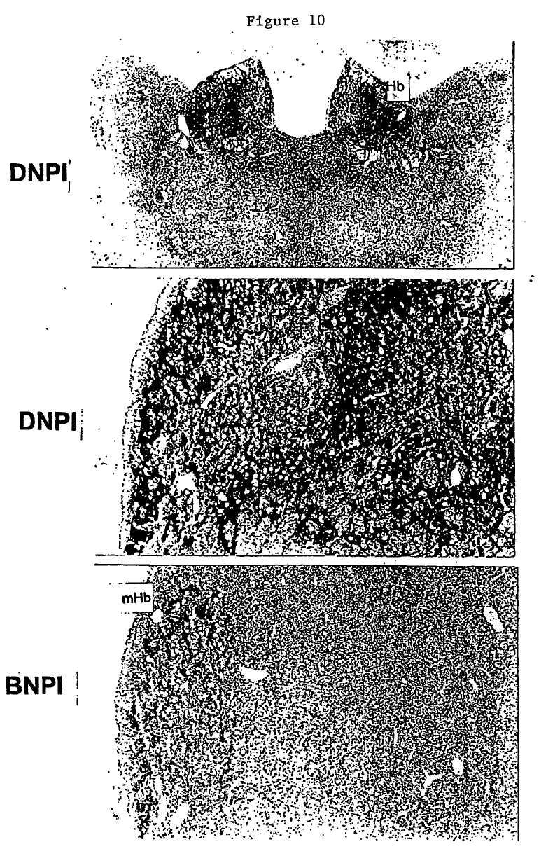

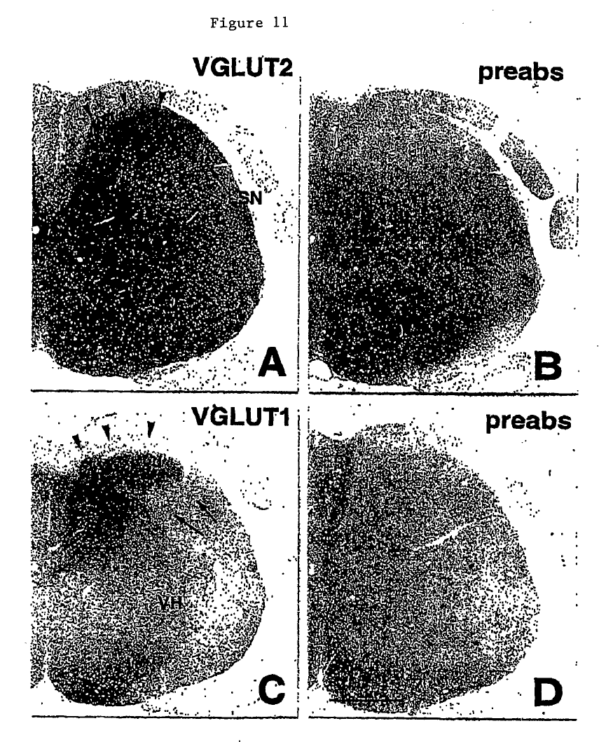

### Example 2h Relating to FIG. 10)

Abundance of DNPI over BNPI in the habenulae (Hb). DNPI is present in the entire habenular complex (low magnification, upper image; high magnification, middle image). BNPI is only in the medial habenular nucleus (mHb lower image, successive section to the middle image).

In the following Examples 2i to 2q and the associated FIGS. 11 to 19, the term VGLUT1 is used for BNPI and VGLUT2 for DNPI. The terms are each completely synonymous, wholly identical in terms of content and relate to the same subject, that is to say VGLUT1=BNPI and VGLUT2=DNPI. “preabs” means pre-absorption with VGLUT1 or VGLUT2 fusion protein prior to the immune staining.

### Example 2i Relating to FIG. 11)

This is a comparison between VGLUT1 and VGLUT2 in respect of their distribution and specificity in the lumbar spinal cord via the immune reactivity.

The adjacent sections A to D are stained alternately with anti-VGLUT2 (A), anti-VGLUT2 pre-absorbed with VGLUT2 fusion protein (B), anti-VGLUT1 (C) and anti-VGLUT1 pre-absorbed with VGLUT1 fusion protein (D). The immune reactions in (A) and (C) are completely pre-absorbed with homologous recombinant fusion protein (B) and (D), which demonstrates the specificity of the immune reaction. The differential distribution pattern in the superficial and deep dorsal horn (A;C) is to be noted. Point-like immune staining for VGLUT2 is to be seen in the superficial dorsal horn (arrows denote in A lamina 1 and substantia gelatinosa), where the immune reactivity with VGLUT1 is minimal (arrows in C). Also to be noted is the accumulation of strongly positive point-like VGLUT1 in the deep dorsal horn, where VGLUT2 is relatively weak. VGLUT2 is present in the lateral spinal nucleus (LSN; arrows in A), where VGLUT1 is only slightly represented (arrows in C). VGLUT2 is strongly represented in lamina X around the central canal, where VGLUT1 is rare. The immune staining of VGLUT1 is weak to moderate in the lateral ventral horn and very thin in the medial ventral horn. A fine point-like VGLUT2 staining is dense and abundant in the ventral horn (VH).

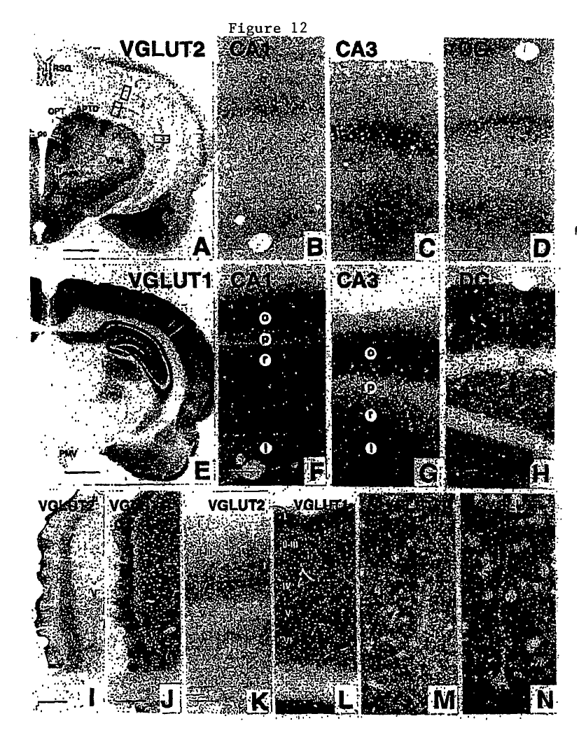

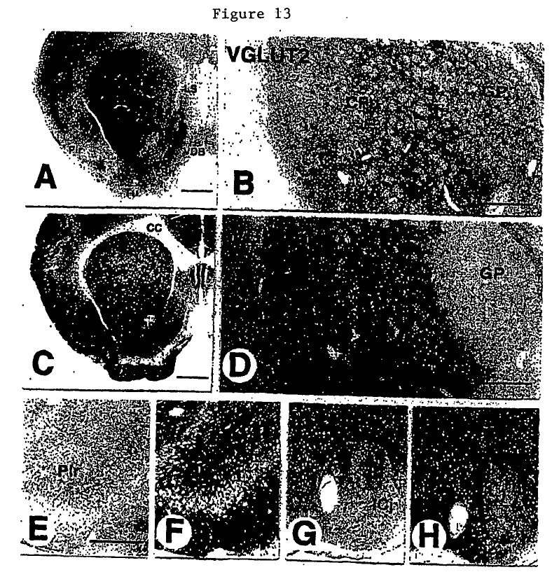

### Example 2j Relating to FIG. 12)

This is a comparison between VGLUT1 and VGLUT2 in respect of their distribution in the forebrain (1).

Pairs of low-power and high-power micrographs of two adjacent frontal sections are alternately stained for VGLUT2 (A-D, I, K, M) and VGLUT1 (E-H, J, L, N). This shows the marked difference and the partially mutual exclusivity in the distribution, density and intensity of VGLUT1 and VGLUT2 immune reactivity (ir) in selected cortical, hippocampal, diencephalic and limbic regions.

Hypothalamus and thalamus (A,E): Point-like VGLUT2-ir is relatively strong over the entire hypothalamus and thalamus, where VGLUT2 shows limited distribution on the hypothalamic, ventral premammillary nucleus (PMV) and on parts of the thalamic nucleus including the lateral posterior thalamic nucleus (LP), the dorsal lateral geniculate nucleus (DLG) and the ventral posteromedial thalamic nucleus (VPM). Olivary pretectal nucleus (OPT); dorsal anterior pretectal nucleus (APTD); precommissural nucleus (Prc).

Cortex (A; E; I; J; K; L; M; N): VGLUT2 staining is moderate in a strip of the neocortex containing lamina IV. It is weak in a neocortical strip containing lamina VI and minimal in the other neocortical layers. Intensive point-like VGLUT1-ir is very highly pronounced in the entire cortex, including the pririform cortex (Pir), and slightly less pronounced in the neocortical band of lamina VI, where moderate VCGLUT2 staining accumulates. The mutual exclusion of VGLUT1 and VGLUT2 staining in the layers of the retrospenial granular cortex (RSG in A and E), which are shown in higher magnification in (I) and (J), is to be noted. The high magnifications M and N from lamina IV in K and L show different densities of the point-like VGLUT1- and VGLUT2-ir in a comparison between immunonegative neuronal cell bodies and processes.

Hippocampus (A, E; B-D, F-H): Thin VGLUT2-ir points are mostly limited to the granular layers (g) of the dentate gyrus (DG) and the pyramidal layer (p) of fields CA1, CA2 and CA3 of the hippocampus. Dense VGLUT1-ir points are very strongly represented over the entire hippocampus with the exception of the granular (g) and pyramidal (p) cell layers. Portions in A marked with rectangles and corresponding portions on adjacent sections in E are shown at higher magnification in B-D and F-H, respectively. The differential distribution and density of the VGLUT1-ir and VGLUT2-ir in the oriens layer (o), the pyramidal layer (p), in the stratum radiatum (r) and the stratum lacunosum molecular (1) of CA1 (B,F) and CA3 (C, G) and in the molecular (m), granular (g) and polymorphous (p) layer of the dentate gyrus (DG) (D,H) are to be noted.

Amygdala complex: A certain overlap is to be found here, but the differential density and intensity of the immune staining of VGLUT1-ir and VGLUT2-ir points in the posterior basomedial amygdaloidal nucleus (BMP), in the lateral amygdaloidal nucleus (La) and in the cortical amygdaloidal nucleus (Co), as well as in the adjacent dorsal endopiriform nucleus (DEn), can clearly be seen.

It is also to be noted that white matter and fiber tracts are VGLUT1-and VGLUT2-negative (posterior commissure (pc), fornix (f), fasciculus retroflexus (fr), mammillothalamic tract (mt).

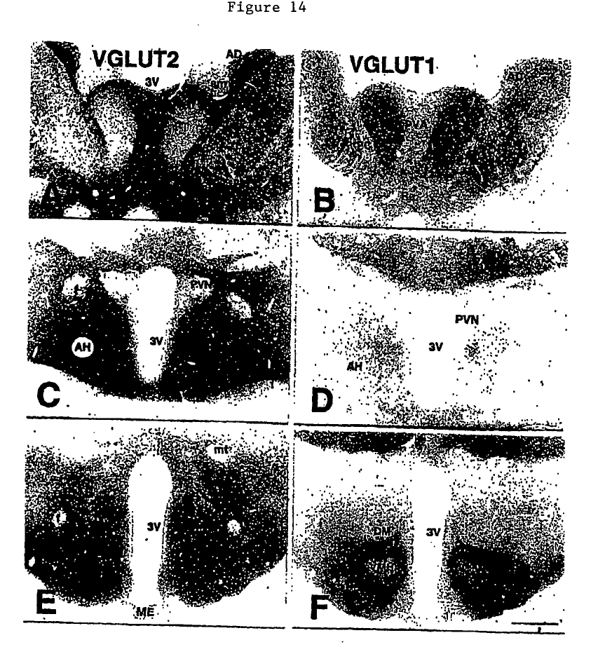

### Example 2k Relating to FIG. 13)

This is a comparison between VGLUT1 and VGLUT2 in respect of their distribution in the forebrain (2).

Pairs of low-power and high-power micrographs of two adjacent frontal sections are stained alternately for VGLUT2 (A-B, E, G) and VGLUT1 (C-D, F, H). This shows the marked difference and the partially mutual exclusivity in the distribution, density and intensity of VGLUT1 and VGLUT2 immune reactivity (ir) in the neocortex (lamina IV, VI), caudate putamen (CPu), globulus pallidum (GP), piriform cortex (Pir), nucleus accumbens core (AcC), nucleus accumbens shell (AcSh), ventral pallidum (VP), olfactory tubercle (Tu), islands of Calleja (ICj), the ventral diagonal band (VDB) and the lateral septum (LS). In the CPu, VGLUT1 is slightly more weakly represented than VGLUT2. VGLUT2 is present in the globus pallidum (GP) (B), where VGLUT1 is almost completely absent (D). In the piriform cortex (Pir) and the islands of Calleja (ICj), the point-like VGLUT1-ir is more strong and more dense than for VGLUT2-ir. The accumulation of weak to moderate VGLUT1-ir in the pyramidal cell layers in (E), where VGLUT2 is almost completely absent (F), is to be noted. Also to be noted is a certain overlap and reciprocity in the staining of VGLUT1 and VGLUT2 in the ICj (G, H), and also the absence of VGLUT1-ir and VGLUT2-ir in the commissural fiber tract/corpus callosum (cc(anterior commissure (ac).

Bar in A, C=1 mm; in B, D=500 μm; in E-H=200 μm.

### Example 21 Relating to Example 14)

This is a comparison between VGLUT1 and VGLUT2 in respect of their distribution in the thalamic and hypothalamic nucleus.

Adjacent frontal sections (A, B) of the diencephalon show the nucleus-specific differential pronounced occurrence of VGLUT2 (A) and VGLUT1 (B) in the thalamus and hypothalamus. The very strong occurrence of VGLUT2-ir (A) in the paraventricular thalamic nucleus (PVA), reuniens thalamic nucleus (Re), reticular thalamic nucleus (Rt), paracentral thalamic nucleus (PC) and anterodorsal thalamic nucleus (AD) is to be noted. VGLUT1-ir (B) is absent almost completely therein or occurs in only a low concentration. VGLUT1 (B) occurs moderately in the posterior thalamic nucleus (PT), where VGLUT2 (A) is rare. VGLUT1 is almost completely absent in the stria medullaris (sm), where VGLUT2-ir is rare.

Adjacent frontal portions C, D of the diencephalon show the frequency of VGLUT2 (C) in the anterior hypothalamic nucleus (AH) but its rarity in the paraventricular nucleus (PVN), and the extreme rarity of VGLUT1-ir in the anterior hypothalamic nucleus (AH) and its total absence in the PVN. Also to be noted is the presence of VGLUT1 (D) in contrast to the absence of CGLUT2 (C) in the ventromedial thalamic nucleus (VM) and the reuniens thalamic nucleus (Re).

Adjacent frontal sections of the hypothalamus (E, F) show the frequency of VGLUT2 in the LH, in the ventromedial thalamic nucleus (VHM) and the dorsomedial thalamic nucleus (DM) and the rarity of VGLUT1 in the core of the VMH but its moderate occurrence in its edge. The weak staining of VGLUT2 in the median eminence (EM) is to be noted. VGLUT1 and VGLUT2 are absent in the fiber tracts of the formix (f) and of the mammillothalamic tract (mt). Third ventricle (3V); bar=500 μm (for A-F).

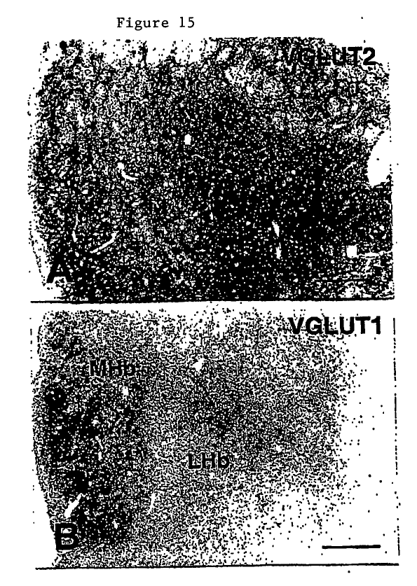

### Example 2m Relating to FIG. 15)

This is a comparison between VGLUT1 and VGLUT2 in respect of their distribution in the epithalamus.

High-power micrographs of adjacent sections, stained alternately for VGLUT2 (A) and VGLUT1 (B), show the excess of VGLUT2 in both the medial habenular nucleus (MHb) and the lateral habenular nucleus (LHb). It is to be noted that VGLUT1-ir is less dense in the MHb than is VGLUT2-ir. CGLUT1 is almost completely absent in the LHb.

Bar=100 μm (for A, B).

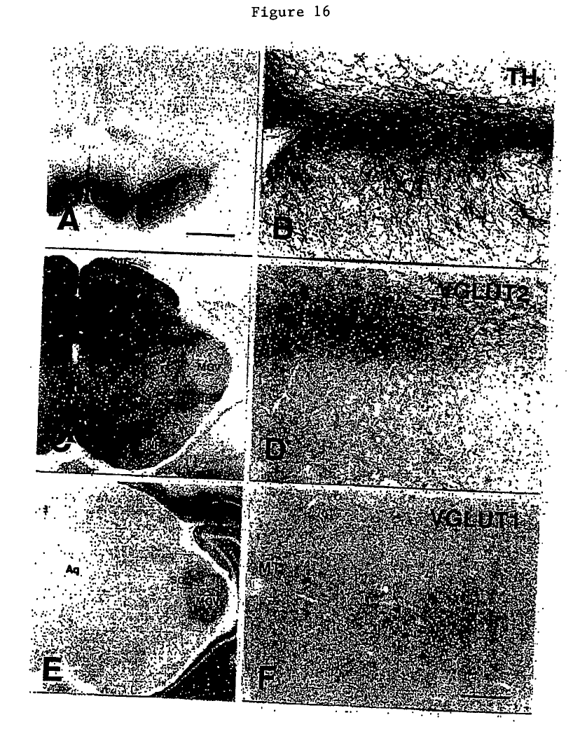

### Example 2n Relating to FIG. 16)

This is a comparison between VGLUT1, tyrosine hydroxylase and VGLUT2 in respect of their distribution in the mesencephalon and metathalamus.

Low-power (A, C, E) and high-power (B, D, F) micrographs of three adjacent sections are stained alternately for tyrosine hydroxylase (TH), VGLUT2 and VGLUT1 and show the differential distribution, density and intensity of the immune staining for VGLUT1 and VGLUT2 in comparison with TH. VGLUT2-ir points are concentrated in the tectum, the highest amounts being found in the superficial grey layer of the superior colliculus (SuG) and smaller amounts being found in the intermediate grey layer of the superior colliculus (InG), whereas these are rare in the optical nucleus layer of the superior colliculus (Op). VGLUT2-ir points are present in the entire tegmentum including the nucleus ruber (R) and the TH-positive pars compacta of the substantia nigra (SNC) and are particularly concentrated in the dorsal periaquaductal grey matter (PAG) and especially in the medial terminal nucleus of the accessory optic tract (MT) as well as in the mediocaudal part of the lateral posterior nucleus (LPMC), in the posterior intralaminar thalamic nucleus (PIL), in the peripenduncular nucleus (PP) and in the suprageniculate thalamic nucleus (SG). VGKUT1-ir is minimal here. VGLUT1 staining is found in moderate amounts in the ventral medial geniculate nucleus (MGV), where VGLUT2 amounts are minimal. VGLUT1 is present in only minimal amounts in the entire tectum, the periaquaductal grey matter and the tegmetum and is almost completely absent in the substantia nigra pars compacta (SNC) and the pars reticulais (SNR). Weak VGLUT2 staining is present in the neuronal perikarya and puncta in the SNR (D, high-powered micrograph from that marked with a rectangle in (C), where VGLUT1 is almost completely absent (corresponding in F)). Mesencephalic aquaduct (Aq).

Bar in A, C, E=1 mm; in B, D, F=200 μm.

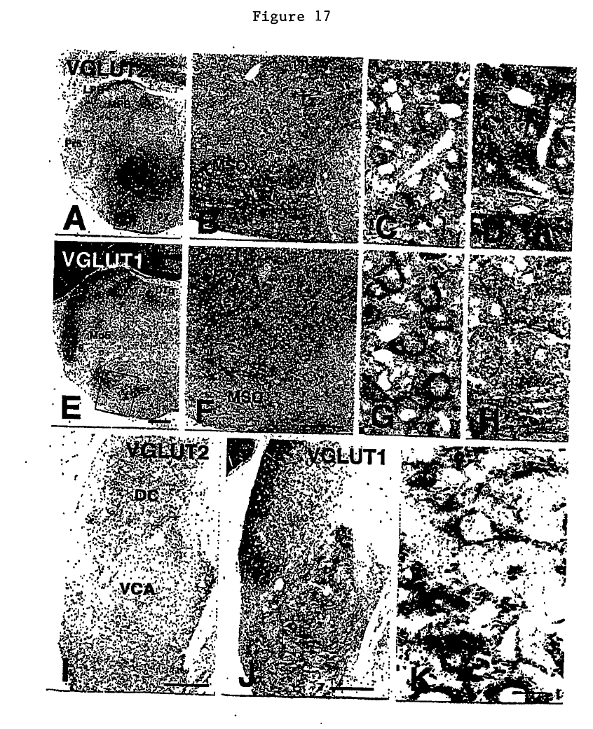

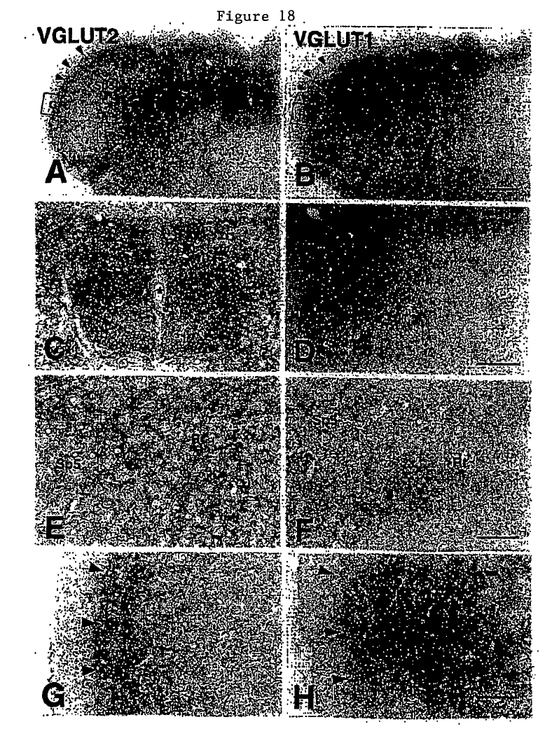

### Example 2o Relating to FIG. 17)

This is a comparison between VGLUT1 and VGLUT2 in respect of their distribution in the pontomedullary brain stem.

Low-power and high-power micrographs of two adjacent frontal sections are stained alternately for VGLUT2 (A-D) and VGLUT1 (E-H). This shows a large amount of point-like VGLUT2 immune reactivity (ir) (B, D) in the medial superior olive (MSO), where VGLUT1-ir is low (F, H). VGLUT2-ir is relatively weak in the nucleus of the trapezoidal body (TZ) (B-C), where VGLUT1-ir is present in a large amount (F-G). It is to be noted that VGLUT1-positive confluent large points clearly surround immunonegative neuronal cell bodies in the TZ (G). Strong VGLUT1-ir points are in the central sensory nucleus of the trigeminal nerve (Pr5), where VGLUT2-ir is very low. Moderately positive VGLUT1 points are present in the motor trigeminal nucleus (Mo5), where VGLUT2-ir is low. VGLUT1-ir and VGLUT2-ir are represented in a small amount in the lateral medial parabrachial nucleus (LBP, MPB). Moderate VGLUT2-ir accumulates in the locus coerulus (LC), where VGLUT1-ir is very low. VGLUT1-ir and VGLUT2-ir are absent in the pyramidal tract (pyr). Adjacent sectors (I, J), stained alternately for VGLUT2 (I) and VGLUT1 (J), show a large amount of point-like VGLUT1 immune reactivity (ir) in the front ventral cochlear nucleus (VCA), where VGLUT2 is in principle totally absent. A high-power micrograph (K) of J shows highly positive VGLUT-ir points, which include immunonegative neuronal cell bodies and processes. VGLUT1-ir points are in a larger number in the dorsal cochlear nucleus (DC) than VGLUT2-ir-positive points.

Bar in A, E=500 μm; in B, F, I, J=200 μm; in C, D, G, H, K=25 μm.

### Example 2p Relating to FIG. 18)

This is a comparison between VGLUT1 and VGLUT2 in respect of their distribution in the lower brain stem.

Low-power and high-power micrographs of two adjacent frontal sections are stained alternately for VGLUT2 (A, C, E, G) and VGLUT1 (B, D, F, H), show a moderate amount of small point-like VGLUT2 immune reactivity (ir) in the superficial spinal trigeminal nucleus (Sp5), which is marked by arrows (A, G), where VGLUT1-ir is in principle totally absent (arrows in B, H). VGLUT2 is present in a moderate amount in the dorsal motor nucleus of the vagus (10), hypoglossal nucleus (12), the reticular formation (Rt) and in the ventral part of the solitary tract (SolV) (A, C, E), where VGLUT1 is in principle totally absent (B, D, F). VGLUT2-ir is very low in the dorsal solitary tract (SolD). The excess VGLUT1 in the deep Sp5 (B, F, H), in the cuneate (Cu) and the grazil nucleus (GR), where VGLUT2-ir is low, is to be noted. Asterisks mark the central canal.

Bar in A, B=500 μm; C, D=200 μm; E, F=100 μm; G, H=100 μm.

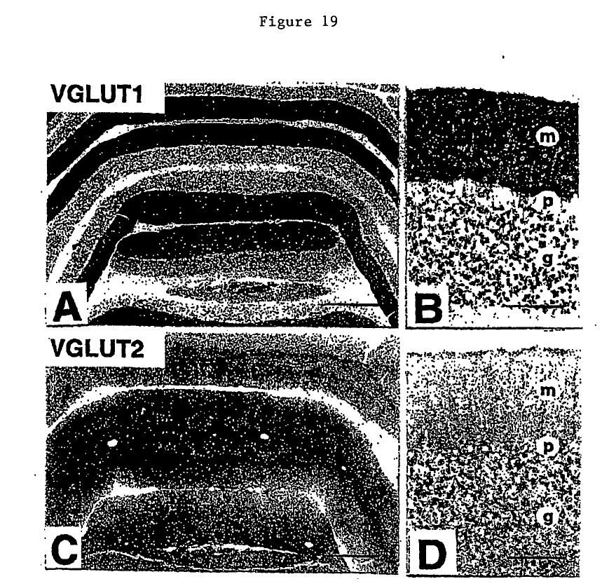

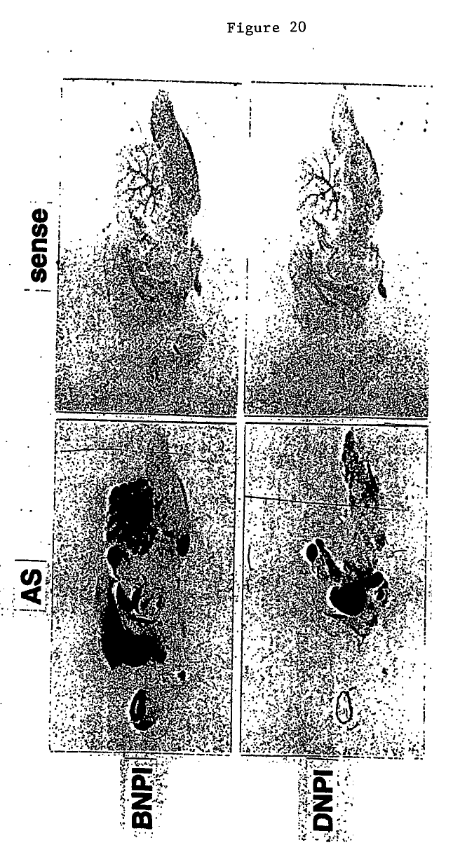

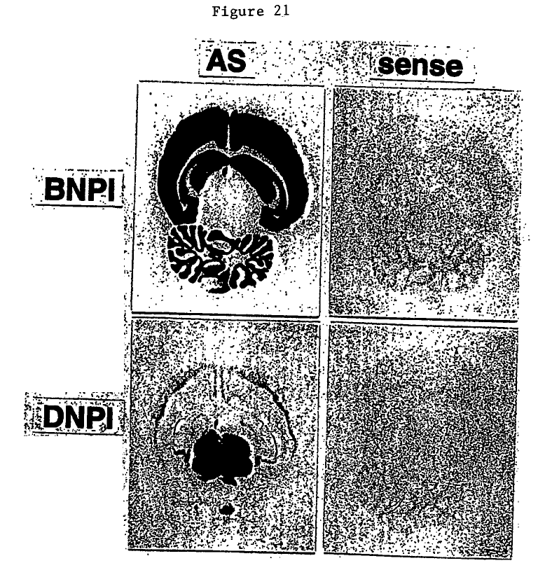

### Example 2q Relating to FIG. 19)

This is a comparison between VGLUT1 and VGLUT2 in respect of their distribution in the cerebellum.

Low-power and high-power micrographs of two adjacent frontal sections, stained alternately for VGLUT2 (A, B) and VGLUT1 (C, D), show an extreme density of intensively stained VGLUT1-positive points in the molecular layer (m), very few VGLUT1 points around somata of the Purkinje cell in the Purkinje cell layer (p) and dense glomeruli-like accumulation of strongly stained confluent VGLUT1 points in the granular layer (g). VGLUT2-ir points are much less dense in the molecular layer, where they are arranged in a strip-like manner. VGLUT2-ir points, which form glomerula-like structures in the glomerular layer (g), are less dense than those which stain for VGLUT1.

Bar in A, C=500 μm; in B, D=100 μm.

### Discussion and Analysis of Example 2 in General:

The differential distribution of BNPI and DNPI in synapses of the primary afferent, spinal trigeminal and supraspinal system is strong evidence that it may be possible selectively to influence sensory functions by selective modulation of DNPI- or BNPI-mediated glutamate transport.

The presence of BNPI and DNP in the DRG suggests that it may be possible to influence peripheral neurogenic inflammations selectively by selective intervention at the DNPI or BNPI target. Their presence in the DRG also indicates an immunomodulatory role and corresponding targeting. The presence of BNPI or DNPI in the sensory vagus or glossopharyngeal ganglion indicates the target for baroafference, chemoafference, cardiovascular or cardiorespiratory function, including asthma, hypertonia, etc., as well as for emesis. The distribution is also of interest for the intestine-brain axis, that is to say regulation of satiation, inflammatory bowel disease or Crohn's disease as well as for autoimmunity in the central or peripheral nervous system, autoimmune diabetes, alcoholic neuropathy, alcohol-induced chronic pancreatitis with neuroproliferation (Fink et al. with Weihe; Neuroscience). The distribution in the CNS and PNS alone makes these targets objects of interest in the other indications already mentioned above.

Another decisive point was, however, that BNPI and DNPI could also be detected in the afferent regions to the sensory regions of the eye and ear which, in combination with the other findings, suggests an important role in visual disturbances, retinitis pigmentosa, optical degeneration, cataracts, detachment of the retina, retinal degeneration, glaucoma or nystagmus, or hearing disorders, tinnitus, Menière's disease, hearing loss, diseases of the organ of hearing and/or balance, or diseases of the auditory canal or vestibular canal.

It is also decisive that the distribution of VGLUT2 (=DNPI) in most regions of the brain and of the spinal cord is complementary and mutually exclusive to the expression of VGLUT1 (=BNPI). Together, the two glutamate transporters could be responsible for the uptake of glutamate by synaptic vesicles of all central glutamatergic neurons.

It has been found that the thalamic and brain stem relay centers of the visual and statoacoustic pathway are driven by differential VGLUT1- and VGLUT2-controlled signals. Thalamic and mesencephalic relay centers of the visual system, such as the colliculus superior and the dorsolateral geniculate nucleus and the medial terminal nucleus of the accessory optic tract, of the related optical system, are specifically VGLUT2-controlled, which suggests that the retinal ganglionic cells, which represent the third neuron of the optical sense, are at least partially coated with VGLUT2. In contrast, the brain stem cochlear, olivary trapezoid and the metathalamic medial geniculate relay center of the auditory pathway receive strong input from VGLUT1-coated glutamatergic synapses.

Different nuclei of the brain stem visual system receive a strong input through VGLUT2 synaptic points. The brain stem of the optical system therefore appears to be supplied solely by VGLUT2 glutamatergic synapses.

The following results and conclusions follow from the tests: DNPI is a new marker for glutamatergic synaptic vesicles, there being 2 different types of neurons or synapses. VGLUT1 and VGLUT2 exhibit a differential distribution pattern.

In summary, the distribution of BNPI and DNPI in the CNS and PNS indicate that they play a role in the various indications already mentioned above, for which pharmaceutically active compounds that attach to these targets are sought with the process according to the invention.

### Example 3

### Implementation of the Screening Process with Measurement of Binding Via the Displacement of a Radioactively Labeled Ligand

A nucleic acid section coding for BPNI is cloned in an expression vector which permits a constitutive expression (e.g., CMV promoter) or an inducible expression in eukaryotic cells. The DNA is introduced into eukaryotic cells (e.g., CHO cells, HEK293 cells or NIH-3T3 cells) by a suitable transfection process, e.g., using Lipofectamine (Roche Diagnostics). The cells are cultivated in the presence of a selection reagent (e.g., Zeocin, hygromycin or neomycin) so that only those cells survive which have taken up the DNA construct and, with a selection of longer duration, have also incorporated it into the genome.

Starting from these cells, membrane fractions which contain a large amount of BNPI and can be used for a binding assay are obtained. This consists of 1.) the membranes containing BNPI, 2.) a radioactively labeled ligand, 3.) a binding buffer (e.g., 50 mM HEPES pH 7.4, 1 mM EDTA) and the ligand to be tested for binding. After incubation of the above-mentioned reaction mixtures (e.g., for 30-60 minutes) at a suitable temperature (in most cases room temperature), the radioactive ligand molecules that have not bonded are filtered off. After addition of a scintillation cocktail, the residual amount of bonded ligand is measured in a β-counter (e.g., Trilux, Wallac). If the test substance exhibits binding to BMPI, this is detected as reduced radioactive incorporation. This process is expediently miniaturized so that it can be carried out on (96-, 384- or 1536-well) microtiter plates in order to carry out the process by means of a robot in the so-called high-throughput screening (HTS) process.

### Example 4

### Implementation of the Screening Process According to the Invention using BNPI and Measurement of the Functional Parameters Changed by the Binding of the Substance

A nucleic acid section coding for BNPI is cloned in an expression vector which permits an inducible expression in prokaryotic cells, e.g., E. coli. The nucleic acid section is so modified thereby that it is expressed as a fusion protein having an additional N- or C-terminal amino acid sequence. With an unchanged function of the BNPI, this sequence should permit purification by a specific process, e.g., glutathione S-transferase fragment which, via binding to glutathione, permits isolation from the protein mixture. After transfection of the bacteria, induction of the gene (e.g., with IPTG in the case of the lac promoter) and opening up of the bacteria, the fusion proteins are purified and used in an in vitro kinase experiment. In this experiment, 5 μg of protein are at 30° C. for 30 minutes in 50 μl kinase buffer (20 mM PIPES, pH 7.0, 5 mM MnCl₂, 7 mM β-mercaptoethanol, 0.4 mM spermine, 10 mM rATP) supplemented with 10 μCi [γ³²P] ATP. Purified histone H1 protein (Sigma) or bacterially expressed GST-NFATc1 fusion protein are added as substrates. After the incubation time, the non-incorporated [γ³²P] ATP is filtered off and the amount of ³²phosphate incorporated is determined by β-scintillation (Trilux, Wallac). In an experiment to trace new BNPI inhibitors, the test substances are incubated concomitantly in this batch and a reduction in 32P incorporation is used as an indicator of an inhibitor. This process is expediently miniaturized so that it can be carried out on (96-, 384- or 1536-well) microtiter plates in order to carry out this process by means of a robot in the so-called high-throughput screening (HTS) process.

### Example 5

### Implementation of the Screening Process According to the Invention using DNPI and Measurement of the Functional Parameters Changed by the Binding of the Substance

The process is carried out as described in Example 4 except that a nucleic acid section coding for DNPI was used instead of a nucleic acid section coding for BNPI.

### Example 6

### Example of a Medicament for the Treatment of Tinnitus or for the Treatment of Motor Neuron Disorders, Containing a Compound According to the Invention—Tablet Formulation

Tablets can be prepared by the direct compression of mixtures of the compound according to the invention with appropriate excipients or by the compression of granules containing the compound (optionally with further excipients). The granules may be prepared either by wet granulation using, for example, aqueous granulation liquids and with subsequent drying of the granules, or by dry granulation, for example by means of compaction.

Direct compression

| | | |
|---|---|---|
| e.g., per tablet: | 25 mg | compound according |
| | | to the invention |
| | 271 mg | LudipressTM (granulate |
| | | for direct tabletting |
| | | consisting of lactose |
| | | monohydrate, povidone |
| | | K30 and crospovidone) |
| | 4 mg | magnesium stearate |
| | 300 mg in total | |

Prepare a homogeneous mixture of the active substance with the excipients and compress the mixture on a tablet press to form tablets having a ø of 10 mm.

Dry granulation

| | | |
|---|---|---|
| e.g., per tablet: | 25 mg | compound according to |
| | | the invention |
| | 166 mg | microcrystalline cellulose |
| | 80 mg | low substituted |
| | | hydroxypropylcellulose |
| | | (J-HPC LH 1 1TM) |
| | 5 mg | highly dispersed |
| | | silicon dioxide |
| | 4 mg | magnesium stearate |
| | 280 mg in total | |

Prepare a homogeneous mixture of the compound with the microcrystalline cellulose and the I-HPC and compact the mixture. After sieving the compressed products, the resulting granules are mixed with magnesium stearate and silicon dioxide and compressed on a tablet press to form tablets having a ø of 9 mm.

Wet granulation

| | | |
|---|---|---|
| e.g., per tablet: | 25 mg | compound according to |
| | | the invention |
| | 205 mg | microcrystalline cellulose |
| | 6 mg | povidone K30 |
| | 10 mg | crospovidone |
| | 4 mg | magnesium stearate |
| | 250 mg in total | |

Prepare a homogeneous mixture of the compound with the microcrystalline cellulose and the crospovidone and granulate the mixture in a granulator with an aqueous solution of the povidone. The moist granules are then granulated and after drying dried in a drying cabinet (50° C.) for 10 hours. The dry granules are sieved together with the magnesium stearate, subjected to a final mixing operation and compressed on a tablet press to form tablets having a ø of 8 mm.

### Example 7

### Example of a Medicament for the Treatment of Tinnitus or for the Treatment of Motor Neuron Disorders, Containing a Compound According to the Invention—Parenteral Solution

1 g of a compound according to the invention is dissolved in 1 liter of water for injection purposes at room temperature and then adjusted to isotonic conditions by addition of NaCl (sodium chloride).

The foregoing description and examples have been set forth merely to illustrate certain embodiments of the invention and are not intended to be limiting. Since modifications of the disclosed embodiments incorporating the spirit and substance of the invention may occur to persons skilled in the art, the invention should be construed broadly to include all variations within the scope of the appended claims and equivalents thereof.

### Literature:

Aihara Y, Mashima H. Onda H. Hisano Setsuji, Kasuya H., Hori T. Yamada S., Tomura H. Yamada Y., Inoue I., Kojima I. and Takeda J. (2000), J. Neurochem. 74: 2622-2625

Akopian A N, Sivilotti L & Wood J N (1995) Nature 379: 257-262

Ausubel F M, Brent R, Kingdton R E, Moore D D, Seidman J G, Smith J A & Struhl K eds. (1190) Current protocols in molecular biology. John Wiley & Sons, Inc. New York, N.Y.

Baba H, Doubell T P, Woolf C J 1999: Peripheral inflammation facilitates Aβ fiber-mediated synaptic input to the substantia gelatinosa of the adult rat spinal cord. J Neurosci 19: 859-867

Bauer D, Muller H, Reich J, Riedel H, Ahrenkiel V, Warthoe P & Strauss M (1993): Identification of differentially expressed mRNA species by an improved display technique (DDRT-PCR) Nucl Acids Res 21: 4272-4280.

Bonini A, Anderson S M, Steiner D F (1997) Molecular cloning and tissue expression of a novel orphan G protein-coupled receptor from rat lung. Biochem Biophys Res Comm 234: 190-193.

Chih-Cheng et al., (1995): A P2X prinoceptor expressed by a subset of sensory neurons. Nature 377: 428-432

Corderre T J, Katz J, Vaccarino A L, Melzack R (1993): Contribution of central plasticity to pathological pain: review of clinical and experimental evidence. Pain 52: 259-285.

Dickenson (1995) Novel pharmacological targets in the treatment of pain. Pain Rev., 2, 1-12.

Dubuisson et al., 1997 Pain, 4: 161-174.

Feng Y & Gregor P (1997) Cloning of a novel member of the G Protein-coupled receptor family related to peptide receptors. Biochem Biophys Res Comm 231: 651-654.

Furukawa T, Yang Y, Nakamoto B, Stamatoyannopoulos G, Papayannopoulou T (1996): Identification of new genes expressed in a human erythroleukemia cell line. Bloods Cell Mol & Dis 22: 11-22.

Gunasekar P G, Kanthasamy, A G, Borowitz J L, Isom G E 1995: NMDA receptor activation produces concurrent generation of nitric oxide and reactive oxygen species: implication for cell death. J Neurochem 65: 2016-2021.

Hawes B E, Fried S, Yao X, Weig B, Graziano M P 1998: Nociceptin (ORL1) and μ-Opioid receptors mediate mitogen-activated protein kinase activation in CHO cells through a Gi-coupled signaling pathway: evidence for distinct mechanisms of agonist-mediated desensitization. J Neurochem 71: 1024-1033.

Hubank M & Schatz D G (1994): Identifying differences in mRNA expression by representational difference analysis of cDNA. Nucl Acids Res 22: 5640-5648. Kluβmann S et al., 1996: Nature Biotechnology 14: 1112-1115.

Li L-Y & Chang K-J 1996: The stimulatory effect of opioids on mitogen-activated protein kinase in Chinese hamster ovary cells transfected to express μ-opioid receptors. Mol Pharm 50: 599-602.

Liang P & Pardee A B 1992: Differential Display of eukaryotic messenger RNA by means of the polymerase chain reaction. Science 257: 967-971.

Methner A, Hermey G, Schinke B, Hermanns-Borgmeyer I (1997): A novel G protein-coupled receptor with homology to neuropeptide and chemoattractant receptors expressed during bone development. Biochem Biophys Res Comm 233: 336-342.

Mohit A A, Martin J H & Miller C A 1995: p493F12 Kinase: a novel MAP kinase expressed in a subset of neurons in the human nervous system. Neuron 14: 67-78.

Poirier G M-C, Pyati J, Wan J S, Erlander M G 1997: Screening differentially expressed cDNA clones obtained by differential display using amplified RNA. Nucleic Acids Research 25: 913-914.

Sambrook J, Fritsch E F & Maniatis T 1989: Molecular Cloning: A Laboratory Manual. Second Edition. Cold Spring Harbor Laboratory Press, Cold Spring Harbor.

Sompayrac L, Jane S, Burn T C, Tenen D G & Danna K J 1995: Overcoming limitations of the mRNA differential display technique. Nucleic Acids Research 23: 4738-4739.

Tal M 1996: A novel antioxidant alleviates heat hyperalgesia in rats with an experimental painful neuropathy. Neurreport 7: 1382-1384.

Tölle TR (1997): Chronischer Schmerz. In: Klinische Neurobiologie, Herdergen T, Tölle T R, Bahr M (eds.): p. 307-336; Spektrum Verlag, Heidelberg.

U.S. Pat. No. 5.262.311

Velculescu V E, Zhang L, Vogelstein B, Kinzler K W (1995): Serial analysis of gene expression. Science 270: 484-487.

Wan J S, Sharp J S et al. (1996): Cloning differentially expressed mRNAs Nature Biotech 14: 1685-1691.

Watson J B & Margulies J E (1993) Differential cDNA screening strategies to identify novel stage-specific proteins in the developing mammalian brain. Dev Neurosci 15: 77-86.

Wilks A F (1989) Two putative protein-tyrosine kinases identified by application of the polymerase chain reaction. Poc Natl Acad Sci USA 86: 1603-1607.

WO 96/34288, Human Brain Sodium-Dependent Inorganic Phosphate Cotransporter, published Oct. 31, 1996; Ni Binhui, Steven M. Paul.

Woolf C J, Shortland P, Coggeshall R E 1992: Peripheral nerve injury triggers central sprouting of myelinated afferents. Nature 355: 75-78.

Zimmermann, M & Herdegen, T (1996): Plasticity of the nervous system at the systemic, cellular and molecular levels: a mechanism of chronic pain and hyperalgesia. Progr Brain Res 110: 233-259.

What is claimed is:

1. A method for identifying pharmaceutically relevant substances having activity in the following indications or effective for the treatment of
visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischaemic attacks), emesis, dizziness, cataracts, arthritis, hyperactivity, developmental disorders, rabies, viral infections, bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, neurodegeneration, Alzheimer's disease, ischaemia; encephalitis; prion disease, Rasmussen's encephalitis, HIV encephalitis, demyelinisation, retinal degeneration, glaucoma, nystagmus, detachment of the retina, diseases of the cerebellum, cerebellar ataxia, diseases of the basal ganglia, diseases of the pallidum, diseases of the organ of hearing or balance, diseases of the auditory canal or vestibular canal, memory disorders, learning disorders, cognitive disorders, stiff man syndrome, restless leg syndrome, anxiety, phobias, sleep disorders; drug dependency, addiction or withdrawal; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease,
said method comprising the steps of:
(a) incubating a test substance
with at least one biomolecule selected from group I, wherein group I consists of:
the protein BNPI or DNPI or a protein comprising SEQ ID NO: 2, 4, 6, 8, 10, 12 or 14 or a protein which is at least 90% homologous thereto or a protein encoded by a polynucleotide comprising SEQ ID NO: 1, 3, 5, 7, 9, 11 or 13 or a polynucleotide which is at least 90% homologous thereto, or a protein encoded by a nucleic acid which binds under stringent conditions to a polynucleotide comprising SEQ ID NO: 1, 3, 5, 7, 9 or 11 or an antisense polynucleotide thereof, or a partial protein of one of the above-mentioned proteins which is at least 10 amino acids long, or
with a cell or a preparation from a cell which has synthesized at least one of the above-mentioned biomolecules of group I, and
(b) measuring the binding of the test substance to the biomolecule of group I or a cell or preparation of a cell which has synthesized at least one of the above-mentioned biomolecules of group I via a change in a known labelled ligand of (i) the biomolecule contained in at least one cell membrane or (ii) the partial protein or protein or via the activity of a labelled test substance bonded thereto, or
measuring at least one functional parameter changed by the binding of the test substance to the biomolecule of group I or a cell or a preparation of a cell which has synthesized at least one of the above-mentioned biomolecules of group I.

2. The method of claim 1, wherein said biomolecule is a partial protein of one of the above-mentioned proteins which is at least 20 amino acids long.

3. The method of claim 1, wherein the cell is manipulated by genetic engineering before step (a).

4. The method of claim 3, wherein the cell manipulation by genetic engineering allows the measurement of at least one functional parameter modified by the binding of the test substance.

5. The method of claim 4, wherein the manipulation by genetic engineering causes expression of a form of a G-protein which is not expressed endogenously in the cell or introduction of a reporter gene.

6. The method of claim 3, wherein the cell is manipulated by genetic engineering so that the cell contains at least one polynucleotide selected from the group consisting of SEQ ID NOS 1, 3, 5, 7, 9, 11 and 13, or a polynucleotide which is at least 90% homologous thereto.

7. The method of claim 6, wherein the polynucleotide is contained in a recombinant DNA construct.

8. The method of claim 3, wherein, after the manipulation by genetic engineering according to claim 3 and before step (a), the cell is cultivated under conditions which allow expression.

9. The method of claim 8, wherein the cell is cultured under selection pressure.

10. The method of claim 1, wherein the cell is an amphibian cell, bacterial cell, yeast cell, insect cell or an immortalized or native mammalian cell.

11. The method of claim 1, wherein the measurement of at least one of the functional parameters modified by the test substance is carried out via measurement of the regulation, inhibition or activation of receptors, ion channels or enzymes.

12. The method of claim 1, wherein the measurement of at least one of the functional parameters modified by the test substance is carried out via measurement of the modification of the gene expression, the ionic environment, the pH, the membrane potential, the enzyme activity or the concentration of the second messenger.

13. The method of claim 1, wherein a first method according to claim 1 is coupled with a second method according to claim 1 such that the measurement values and results of the first method with regard to the substance to be measured are compared with the measurement values and results of the second method with regard to the substance to be measured, wherein one of the two methods is the main method, wherein, in step (a) of said main method, the test substance is incubated with either
a biomolecule selected from group II, wherein group II consists of the protein BNPI or a protein comprising SEQ ID NO: 2, 4, 6 or 8 or a protein which is at least 90% homologous thereto or a protein encoded by a polynucleotide comprising SEQ ID NO: 1, 3, 5 or 7 or by a polynucleotide which is at least 90% homologous thereto, or a protein encoded by a nucleic acid which binds under stringent conditions to a polynucleotide comprising SEQ ID NO: 1, 3, 4, or 7 or the polynucleotides thereof, or a partial protein of one of the above-mentioned proteins which is at least 10 amino acids long,
or a cell or a preparation from a cell that has synthesized at least one of the above-mentioned proteins or partial proteins, or biomolecules of group II,
or a biomolecule selected from group III, wherein group III consists of the protein DNPI or a protein comprising SEQ ID NO: 10, 12 or 14 or a protein which is at least 90% homologous thereto or a protein encoded by a polynucleotide comprising SEQ ID NO: 9, 11 or 13 or by a polynucleotide which is at least 90% homologous thereto, or a protein encoded by a nucleic acid which binds under stringent conditions to a polynucleotide comprising SEQ ID NO: 9, 11 or 13 or an antisense polynucleotide thereof, or a partial protein of one of the above-mentioned proteins which is at least 10 amino acids long,
or a cell or a preparation from a cell that has synthesized at least one of the above-mentioned proteins or partial proteins, or biomolecules of group III,
and
wherein, in a secondary method, in step (a) the test substance is incubated with a biomolecule from group I or with a biomolecule selected from group II or group III wherein the biomolecule selected from group II or group III is selected from a group which differs from the group the biomolecule with which the test substance is incubated in the main method.

14. The method of claim 13 wherein the partial protein of group II or the partial protein of group III has a length of at least 20 amino acids.

15. The method of claim 1, wherein the substances to be identified are selected from the group of substances having activity in the following indications or effective for the treatment of
visual disturbances, retinitis pigmentosa, optical degeneration, hearing disorders, tinnitus, Menière's disease, hearing loss, amyotrophic lateral sclerosis, neuralgia, weight regulation, obesity, Parkinson's disease, cataracts, viral infections or bacterial infections, diabetic neuropathy, autoimmune diabetes, alcoholic neuropathy, HIV-neuroAIDS; retinal degeneration, glaucoma, nystagmus, detachment of the retina, diseases of the organ of hearing or balance, diseases of the auditory canal or vestibular canal, sleep disorders, drug dependency, addiction or withdrawal; neuroinflammation, insomnia, for adjuvant therapy by electrostimulation of the nucleus subthalamicus in Parkinson's disease; or substances for the treatment of diseases of the spinal motor neuron, muscular atrophies or muscular dystrophies.

16. The method of claim 1, wherein the substances to be identified are selected from the group of substances having activity in the following indications or effective for the treatment of
visual disturbances, retinitis pigmentosa, optical degeneration, hearing disorders, tinnitus, Menière's disease, hearing loss, amyotrophic lateral sclerosis, weight regulation, obesity, cataracts, viral infections or bacterial infections, retinal degeneration, glaucoma, nystagmus, detachment of the retina, diseases of the organ of hearing or balance, diseases of the auditory canal or vestibular canal, drug dependency, addiction or withdrawal; neuroinflammation; or substances for the treatment of diseases of the spinal motor neuron, muscular atrophies or muscular dystrophies.

17. The method of claim 1, wherein the substances to be identified are selected from the group of substances having activity in the following indications or effective for the treatment of
visual disturbances, retinitis pigmentosa, optical degeneration, cataracts, detachment of the retina, retinal degeneration, glaucoma or nystagmus; or
hearing disorders, tinnitus, Menière's disease, hearing loss, diseases of the organ of hearing or balance or diseases of the auditory canal or vestibular canal; or
substances for the treatment of diseases of the spinal motor neuron, muscular atrophies or muscular dystrophies.

18. A compound identifiable, by the method of claim 1, as a pharmaceutically relevant substance having activity in at least one of the indications according to claim 1.

19. A compound according to claim 18, wherein said compound is a low molecular weight compound.

20. A method of treating a mammal suffering from visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischaemic attacks), emesis, dizziness, cataracts, arthritis, hyperactivity, developmental disorders, rabies, viral infections or bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, neurodegeneration, Alzheimer's disease, ischaemia; encephalitis; prion disease, Rasmussen's encephalitis, HIV encephalitis, demyelinisation, retinal degeneration, glaucoma, nystagmus, detachment of the retina, diseases of the cerebellum, cerebellar ataxia, diseases of the basal ganglia, diseases of the pallidum, diseases of the organ of hearing or balance, diseases of the auditory canal or vestibular canal, memory disorders, learning disorders, cognitive disorders, stiff man syndrome, restless leg syndrome, anxiety, phobias, sleep disorders; drug dependency, addiction or withdrawal; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease, comprising administering to said mammal a therapeutically effective amount of:
a. a polynucleotide which codes for BNPI or DNPI or a polynucleotide which is at least 90% homologous to one of the nucleotide sequences comprising SEQ ID NO: 1, 3, 5, 7, 9, 11 or 13,
b. a polynucleotide of a ribozyme or other DNA enzyme or of a catalytic RNA or DNA which contains a nucleotide sequence which is capable of binding specifically to one of the polynucleotides listed under point a),
c. a vector containing a polynucleotide according to one of points a) or b),
d. BNPI or DNPI or a protein comprising SEQ ID NO: 2, 4, 6, 8, 10, 12 or 14 or a protein which is at least 90% homologous thereto, or a protein encoded by a polynucleotide comprising SEQ ID NO: 1, 3, 5, 7, 9, 11 or 13 or a polynucleotide which is at least 90% homologous thereto, or a protein encoded by a nucleic acid which binds under stringent conditions to a polynucleotide comprising SEQ ID NO: 1, 3, 5, 7, 9, 11 or 13 or an antisense polynucleotide thereof, or a partial protein of one of the above-mentioned proteins which is at least 10 amino acids long,
e. an antibody against one of the proteins or partial proteins according to point d),
f. a cell containing a polynucleotide according to one of points a) or b), a vector according to point c), a protein or partial protein according to point d) or an antibody according to point e),
g. a compound according to claim 18, or
h. an active substance which binds to a protein or partial protein according to point d), and
a pharmaceutically acceptable carrier or auxiliary substance.

21. The method of claim 20, wherein said polynucleotide according to point a) is a DNA or RNA.

22. The method of claim 20, wherein said polynucleotide according to point b) is an antisense polynucleotide, a peptide nucleic acid, a DNA enzyme or a ribozyme.

23. The method of claim 20, wherein said vector according to point c) is an expression vector, a vector derived from a virus or a vector containing at least one LTR, poly A, promoter or ORI sequence.

24. The method of claim 20, wherein said vector according to point c) is an adenovirus, adeno-associated virus or herpes virus.

25. The method of claim 20, wherein said protein or partial protein according to point d) has been post-translationally modified.

26. The method of claim 25, wherein said protein or partial protein according to point d) has been glycosylated, phosphorylated, amidated, methylated, acetylated, ADP-ribosylated, hydroxylated, provided with a membrane anchor, cleaved or shortened.

27. The method of claim 20, wherein said antibody according to point e) is a monoclonal or polyclonal antibody.

28. The method of claim 20, wherein said cell according to point f) is an amphibian cell, bacterial cell, yeast cell, insect cell or an immortalized or native mammalian cell.

29. The method of claim 20, wherein said active substance according to point h) is a low molecular weight active substance.

30. A method of providing gene therapy to a mammal, said method comprising administering to said mammal a therapeutic amount of:
a. a polynucleotide which codes for BNPI or DNPI or a polynucleotide which is at least 90% homologous to a nucleotide sequence comprising SEQ ID NO: 1, 3, 5, 7, 9, 11 or 13,
b. a polynucleotide of a ribozyme or other DNA enzyme or of a catalytic RNA or DNA which contains a nucleotide sequence which is capable of binding specifically to one of the polynucleotides listed under point a),
c. a vector containing a polynucleotide according to one of points a) or b),
f. a cell containing a polynucleotide according to one of points a) or b) or a vector according to point c).

31. The method of claim 30, wherein the gene therapy is in vivo gene therapy.

32. The method of claim 30, wherein the gene therapy is in vitro gene therapy.

33. The method of claim 30, wherein said polynucleotide according to point a) is a DNA or RNA.

34. The method of claim 30, wherein said polynucleotide according to point b) is an antisense polynucleotide, a peptide nucleic acid, a DNA enzyme or a ribozyme.

35. The method of claim 30, wherein said vector according to point c) is an expression vector, a vector derived from a virus or a vector containing at least one LTR, poly A, promoter or ORI sequence.

36. The method of claim 30, wherein said vector according to point c) is an adenovirus, adeno-associated virus or herpes virus.

37. The method of claim 30, wherein said cell according to point f) is an amphibian cell, bacterial cell, yeast cell, insect cell or an immortalized or native mammalian cell.

38. The method of claim 30, wherein said gene therapy has activity in the following indications or is effective for the treatment of
visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischaemic attacks), emesis, dizziness, cataracts, arthritis, hyperactivity, developmental disorders, rabies, viral infections, bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, neurodegeneration, Alzheimer's disease, ischaemia; encephalitis; prion disease, Rasmussen's encephalitis, HIV encephalitis, demyelinisation, retinal degeneration, glaucoma, nystagmus, detachment of the retina, diseases of the cerebellum, cerebellar ataxia, diseases of the basal ganglia, diseases of the pallidum, diseases of the organ of hearing or balance, diseases of the auditory canal or vestibular canal, memory disorders, learning disorders, cognitive disorders, stiff man syndrome, restless leg syndrome, anxiety, phobias, sleep disorders; drug dependency, addiction or withdrawal; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease.

39. A method of diagnosing a condition selected from:
visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischaemic attacks), emesis, dizziness, cataracts, arthritis, hyperactivity, developmental disorders, rabies, viral infections, bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, neurodegeneration, Alzheimer's disease, ischaemia; encephalitis; prion disease, Rasmussen's encephalitis, HIV encephalitis, demyelinisation, retinal degeneration, glaucoma, nystagmus, detachment of the retina, diseases of the cerebellum, cerebellar ataxia, diseases of the basal ganglia, diseases of the pallidum, diseases of the organ of hearing or balance, diseases of the auditory canal or vestibular canal, memory disorders, learning disorders, cognitive disorders, stiff man syndrome, restless leg syndrome, anxiety, phobias, sleep disorders; drug dependency, addiction or withdrawal; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, comprising administering an effective amount of a diagnostic agent comprising:
a. a polynucleotide which codes for BNPI or DNPI or a polynucleotide which is at least 90% homologous to one of the nucleotide sequences comprising SEQ ID NO: 1, 3, 5, 7, 9, 11 or 13,
b. a polynucleotide of a ribozyme or other DNA enzyme or of a catalytic RNA or DNA which contains a nucleotide sequence which is capable of binding specifically to one of the polynucleotides listed under point a),
c. a vector containing a polynucleotide according to one of points a) or b),
d. BNPI or DNPI or a protein comprising SEQ ID NO: 2, 4, 6, 8, 10, 12 or 14 or a protein which is at least 90% homologous thereto, or a protein encoded by a polynucleotide comprising SEQ ID NO: 1, 3, 5, 7, 9, 11 or 13 or a polynucleotide which is at least 90% homologous thereto, or a protein encoded by a nucleic acid which binds under stringent conditions to a polynucleotide comprising SEQ ID NO: 1, 3, 5, 7, 9, 11 or 13 or an antisense polynucleotide thereof, or a partial protein of one of the above-mentioned proteins which is at least 10 amino acids long,
e. an antibody against one of the proteins or partial proteins according to point d),
f. a cell containing a polynucleotide according to one of points a) or b), a vector according to point c), a protein or partial protein according to point d) or an antibody according to point e),
g. a compound according to claim 18, or
h. an active substance which binds to a protein or partial protein according to point d).

40. The method of claim 39, wherein said polynucleotide according to point a) is a DNA or RNA.

41. The method of claim 39, wherein said polynucleotide according to point b) is an antisense polynucleotide, a peptide nucleic acid, a DNA enzyme or a ribozyme.

42. The method of claim 39, wherein said vector according to point c) is an expression vector, a vector derived from a virus or a vector containing at least one LTR, poly A, promoter or ORI sequence.

43. The method of claim 39, wherein said vector according to point c) is an adenovirus, adeno-associated virus or herpes virus.

44. The method of claim 39, wherein said protein or partial protein according to point d) has been post-translationally modified.

45. The method of claim 44, wherein said protein or partial protein according to point d) has been glycosylated, phosphorylated, amidated, methylated, acetylated, ADP-ribosylated, hydroxylated, provided with a membrane anchor, cleaved or shortened.

46. The method of claim 39, wherein said antibody according to point e) is a monoclonal or polyclonal antibody.

47. The method of claim 39, wherein said cell according to point f) is an amphibian cell, bacterial cell, yeast cell, insect cell or an immortalized or native mammalian cell.

48. The method of claim 39, wherein said active substance according to point h) is a low molecular weight active substance.

49. A method of screening for pharmaceutically relevant substances, said substances having activity in the following indications, or being effective for the treatment of:
visual disturbances, retinitis pigmentosa, optical degeneration, hearing disorders, tinnitus, Menière's disease, hearing loss, schizophrenia, manias, depression, stroke, cerebral trauma, paraplegia, amyotrophic lateral sclerosis, neuralgia, weight regulation, obesity, anorexia nervosa, epilepsy, hemiballism, Huntington's chorea, stress, Parkinson's disease, TIA (transient ischaemic attacks), emesis, dizziness, cataracts, arthritis, hyperactivity, developmental disorders, rabies, viral infections, bacterial infections, influenza, malaria, Creutzfeldt-Jacob disease, inflammatory bowel disease, Crohn's disease, cardiovascular and cardiorespiratory functional disorders, hypertonia, disorders of baroafference or chemoafference, toxoplasmosis, asthma, autoimmunity in the central and peripheral nervous system, diabetic neuropathy, autoimmune diabetes, alcoholic neuropathy, HIV-neuroAIDS; disorders of the autonomous nervous system, disorders of the nervous system of the digestive tract, oversensitivity, neurodegeneration, Alzheimer's disease, ischaemia; encephalitis; prion disease, Rasmussen's encephalitis, HIV encephalitis, demyelinisation, retinal degeneration, glaucoma, nystagmus, detachment of the retina, diseases of the cerebellum, cerebellar ataxia, diseases of the basal ganglia, diseases of the pallidum, diseases of the organ of hearing or balance, diseases of the auditory canal or vestibular canal, memory disorders, learning disorders, cognitive disorders, stiff man syndrome, restless leg syndrome, anxiety, phobias, sleep disorders; drug dependency, addiction or withdrawal; hepatoencephalopathy with alcohol intoxication, hepatoencephalopathy without alcohol intoxication, diseases of neurotoxicological origin, diseases of the spinal motor neuron, muscular atrophies, muscular dystrophies, diseases of the posterior funiculus, alcoholic neuropathies, neuroinflammation, disturbances in the state of mind in the case of infections or fever, stress, taste disorders, food allergies, Chinese restaurant syndrome, aggression, paranoia, brain concussion, neuroendocrine disorders, Tourrette's syndrome, cerebrovascular spasms, neuronal apoptosis, neurodegeneration, neuronal necrosis, astrocytosis, burn-out syndrome, sudden infant death, heart attack, insomnia, retrograde amnesia, multiple sclerosis, jet lag, disorders of sexual function, or having activity for promoting microglia activation, learning, cognition or memory, for neuroprotection, for the liquor diagnosis of neurostatic diseases or for adjuvant therapy by electrostimulation of the nucleus subthalamicus in Parkinson's disease.
said method comprising the steps of:
(a) incubating a test substance with at least one biomolecule selected from group I, wherein group I consists of:
a. a polynucleotide which codes for BNPI or DNPI or a polynucleotide which is at least 90% homologous to one of the nucleotide sequences comprising SEQ ID NO: 1, 3, 5, 7, 9, 11 or 13,
b. a polynucleotide of a ribozyme or other DNA enzyme or of a catalytic RNA or DNA which contains a nucleotide sequence which is capable of binding specifically to one of the polynucleotides listed under point a),
c. a vector containing a polynucleotide according to one of points a) or b),
d. BNPI or DNPI or a protein comprising SEQ ID NO: 2, 4, 6, 8, 10, 12 or 14 or a protein which is at least 90% homologous thereto, or a protein encoded by a polynucleotide comprising SEQ ID NO: 1, 3, 5, 7, 9, 11 or 13 or a polynucleotide which is at least 90% homologous thereto, or a protein encoded by a nucleic acid which binds under stringent conditions to a polynucleotide comprising SEQ ID NO: 1, 3, 5, 7, 9, 11 or 13 or an antisense polynucleotide thereof, or a partial protein of one of the above-mentioned proteins which is at least 10 amino acids long,
e. an antibody against one of the proteins or partial proteins according to point d), or
a cell or a preparation of a cell containing a polynucleotide according to one of points a) or b), a vector according to point c), a protein or partial protein according to point d) or an antibody according to point e), and
(b) measuring the binding of the test substance to the biomolecule of group I, or cell or preparation of a cell containing a polynucleotide according to one of points a) or b), a vector according to point c), a protein or partial protein according to point d) or an antibody according to point e), via a change in a known labelled ligand of (i) the biomolecule contained in at least one cell membrane or (ii) the partial protein or protein or via the activity of a labelled test substance bonded thereto, or
measuring at least one functional parameter changed by the binding of the test substance to the biomolecule of group I or cell or preparation of a cell containing a polynucleotide according to one of points a) or b), a vector according to point c), a protein or partial protein according to point d) or an antibody according to point e).

50. The method of claim 49, wherein said polynucleotide according to point a) is a DNA or RNA.

51. The method of claim 49, wherein said polynucleotide according to point b) is an antisense polynucleotide, a peptide nucleic acid, a DNA enzyme or a ribozyme.

52. The method of claim 49, wherein said vector according to point c) is an expression vector, a vector derived from a virus or a vector containing at least one LTR, poly A, promoter or ORI sequence.

53. The method of claim 49, wherein said vector according to point c) is an adenovirus, adeno-associated virus or herpes virus.

54. The method of claim 49, wherein said protein or partial protein according to point d) has been post-translationally modified.

55. The method of claim 54, wherein said protein or partial protein according to point d) has been glycosylated, phosphorylated, amidated, methylated, acetylated, ADP-ribosylated, hydroxylated, provided with a membrane anchor, cleaved or shortened.

56. The method of claim 49, wherein said antibody according to point e) is a monoclonal or polyclonal antibody.

57. The method of claim 49, wherein said a cell or a preparation of a cell containing a polynucleotide according to one of points a) or b), a vector according to point c), a protein or partial protein according to point d) or an antibody according to point e), is an amphibian cell, bacterial cell, yeast cell, insect cell or an immortalized or native mammalian cell or is a preparation from one of the foregoing cell types.

58. The method of claim 49, wherein said substances have activity in the following indications, or are effective for the treatment of:
visual disturbances, retinitis pigmentosa, optical degeneration, hearing disorders, tinnitus, Menière's disease, hearing loss, amyotrophic lateral sclerosis, neuralgia, weight regulation, obesity, Parkinson's disease, cataracts, viral infections or bacterial infections, diabetic neuropathy, autoimmune diabetes, alcoholic neuropathy, HIV-neuroAIDS; retinal degeneration, glaucoma, nystagmus, detachment of the retina, diseases of the organ of hearing and/or balance, diseases of the auditory canal or vestibular canal, sleep disorders, drug dependency, addiction and withdrawal, especially in the case of alcohol, nicotine, opiates, Ecstasy or cocaine; neuroinflammation, insomnia, for adjuvant therapy by electrostimulation of the nucleus subthalamicus in Parkinson's disease; or diseases of the spinal motor neuron, muscular atrophies or muscular dystrophies.

59. The method of claim 49, wherein said substances have activity in the following indications, or are effective for the treatment of:
visual disturbances, retinitis pigmentosa, optical degeneration, hearing disorders, tinnitus, Menière's disease, hearing loss, amyotrophic lateral sclerosis, weight regulation, obesity, cataracts, viral infections or bacterial infections, retinal degeneration, glaucoma, nystagmus, detachment of the retina, diseases of the organ of hearing and/or balance, diseases of the auditory canal or vestibular canal, drug dependency, addiction and withdrawal, especially in the case of alcohol, nicotine, opiates, Ecstasy or cocaine; neuroinflammation; or diseases of the spinal motor neuron, muscular atrophies or muscular dystrophies.

60. The method of claim 49, wherein said substances have activity in the following indications, or are effective for the treatment of:
visual disturbances, retinitis pigmentosa, optical degeneration, cataracts, detachment of the retina, retinal degeneration, glaucoma or nystagmus; or
hearing disorders, tinnitus, Menière's disease, hearing loss, diseases of the organ of hearing or balance, or diseases of the auditory canal or vestibular canal; or
diseases of the spinal motor neuron, muscular atrophies or muscular dystrophies.

61. The method of claim 49, wherein said emesis is hyperemesis, said oversensitivity is glutamate-mediated sensitivity, said neurodegeneration is related to Alzheimer's disease, said encephalitis is viral or bacterial encephalitis, said demyelinisation is related to multiple sclerosis, said drug dependency, addiction or withdrawal is related to alcohol, nicotine, opiates, Ecstasy or cocaine, or said disorder of sexual function is impotence or priapism.

62. The method of claim 1, wherein said emesis is hyperemesis, said oversensitivity is glutamate-mediated sensitivity, said neurodegeneration is related to Alzheimer's disease, said encephalitis is viral or bacterial encephalitis, said demyelinisation is related to multiple sclerosis, said drug dependency, addiction or withdrawal is related to alcohol, nicotine, opiates, Ecstasy or cocaine, or said disorder of sexual function is impotence or priapism.

63. The method of claim 20, wherein said emesis is hyperemesis, said oversensitivity is glutamate-mediated sensitivity, said neurodegeneration is related to Alzheimer's disease, said encephalitis is viral or bacterial encephalitis, said demyelinisation is related to multiple sclerosis, said drug dependency, addiction or withdrawal is related to alcohol, nicotine, opiates, Ecstasy or cocaine, or said disorder of sexual function is impotence or priapism.

64. The method of claim 38, wherein said emesis is hyperemesis, said oversensitivity is glutamate-mediated sensitivity, said neurodegeneration is related to Alzheimer's disease, said encephalitis is viral or bacterial encephalitis, said demyelinisation is related to multiple sclerosis, said drug dependency, addiction or withdrawal is related to alcohol, nicotine, opiates, Ecstasy or cocaine, or said disorder of sexual function is impotence or priapism.

65. The method of claim 39, wherein said emesis is hyperemesis, said oversensitivity is glutamate-mediated sensitivity, said neurodegeneration is related to Alzheimer's disease, said encephalitis is viral or bacterial encephalitis, said demyelinisation is related to multiple sclerosis, said drug dependency, addiction or withdrawal is related to alcohol, nicotine, opiates, Ecstasy or cocaine, or said disorder of sexual function is impotence or priapism.


SEQUENCE LISTING (14 sequences)

SEQ ID NO: 1 - DNA, 2366 bp, Homo sapiens
ccggcggcaggagccgccaccatggagttccgccaggaggagtttcggaagctagcgggtcgtgctctcgggaagctgcaccgccttctggagaagcggcaggaaggcgcggagacgctggagctgagtgcggatgggcgcccggtgaccacgcagacccgggacccgccggtggtggactgcacctgcttcggcctccctcgccgctacattatcgccatcatgagtggtctgggcttctgcatcagctttggcatccgctgcaacctgggcgtggccatcgtctccatggtcaataacagcacgacccaccgcgggggccacgtggtggtgcagaaagcccagttcagctgggatccagagactgtcggcctcatacacggctcctttttctggggctacattgtcactcagattccaggaggatttatctgtcaaaaatttgcagccaacagagttttcggctttgctattgtggcaacatccactctaaacatgctgatcccctcagctgcccgcgtccactatggctgtgtcatcttcgtgaggatcctgcaggggttggtagagggggtcacataccccgcctgccatgggatctggagcaaatgggccccacccttagaacggagtcgcctggcgacgacagccttttgtggttcctatgctggggcggtggtcgcgatgcccctcgccggggtccttgtgcagtactcaggatggagctctgttttctacgtctacggcagcttcgggatcttctggtacctgttctggctgctcgtctcctacgagtcccccgcgctgcaccccagcatctcggaggaggagcgcaagtacatcgaggacgccatcggagagagcgcgaaactcatgaaccccctcacgaagtttagcactccctggcggcgcttcttcacgtctatgccagtctatgccatcatcgtggccaacttctgccgcagctggacgttctacctgctgctcatctcccagcccgcctacttcgaagaagtgttcggcttcgagatcagcaaggtaggcctggtgtccgcgctgccccacctggtcatgaccatcatcgtgcccatcggcggccagatcgcggacttcctgcggagccgccgcatcatgtccaccaccaacgtgcgcaagttgatgaactgcggaggcttcggcatggaagccacgctgctgttggtggtcggctactcgcactccaagggcgtggccatctccttcctggtcctagccgtgggcttcagcggcttcgccatctctgggttcaacgtgaaccacctggacatagccccgcgctacgccagcatcctcatgggcatctccaacggcgtgggcacactgtcgggcatggtgtgccccatcatcgtgggggccatgactaagcacaagactcgggaggagtggcagtacgtgttcctaattgcctccctggtgcactatggaggtgtcatcttctacggggtctttgcttctggagagaagcagccgtgggcagagcctgaggagatgagcgaggagaagtgtggcttcgttggccatgaccagctggctggcagtgacgacagcgaaatggaggatgaggctgagcccccgggggcaccccctgcacccccgccctcctatggggccacacacagcacatttcagccccccaggcccccaccccctgtccgggactactgaccatgtgcctcccactgaatggcagtttccaggacctccattccactcatctctggcctgagtgacagtgtcaaggaaccctgctcctctctgtcctgcctcaggcctaagaagcactctcccttgttcccagtgctgtcaaatcctctttccttcccaattgcctctcaggggtagtgaagctgcagactgacagtttcaaggatacccaaattcccctaaaggttccctctccacccgttctgcctcagtggtttcaaatctctcctttcagggctttatttgaatggacagttcgacctcttactctctcttgtggttttgaggcacccacaccccccgctttcctttatctccagggactctcaggctaacctttgagatcactcagctcccatctcctttcagaaaaattcaaggtcctcctctagaagtttcaaatctctcccaactctgttctgcatcttccagattggtttaaccaattactcgtccccgccattccagggattgattctcaccagcgtttctgatggaaaatggcggtttcaagtccccgattccgtgcccacttcacatctcccctaccagcagattctgcgaaagcaccaaatttctcaagaccctcttctccctagcttagcataatgtctggggaaaca

SEQ ID NO: 2 - protein, 560 aa, Homo sapiens
MEFRQEEFRKLAGRALGKLHRLLEKRQEGAETLELSADGRPVTTQTRDPPVVDCTCFGLPRRYIIAIMSGLGFCISFGIRCNLGVAIVSMVNNSTTHRGGHVVVQKAQFSWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPLTKFSTPWRRFFTSMPVYAIIVANFCRSWTFYLLLISQPAYFEEVFGFEISKVGLVSALPHLVMTIIVPIGGQIADFLRSRRIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDDSEMEDEAEPPGAPPAPPPSYGATHSTFQPPRPPPPVRDY

SEQ ID NO: 3 - DNA, 2716 bp, Homo sapiens
cgataagcttgatatcgaattccggactcttgctcgggcgccttaacccggcgttcggttcatcccgcagcgccagttctgcttaccaaaagtggcccactaggcactcgcattccacgcccggctccacgccagcgagccgggcttcttacccatttaaagtttgagaataggttgagatcgtttcggccccaagacctctaatcattcgctttaccggataaaactgcgtggcgggggtgcgtcgggtctgcgagagcgccagctatcctgagggaaacttcggagggaaccagctactagatggttcgattagtctttcgcccctatacccaggtcggacgaccgatttgcacgtcaggaccgctacggacctccaccagagtttcctctggcttcgccctgcccaggcgatcggcgggggggacccgcggggtgaccggcggcaggagccgccaccatggagttccgccaggaggagtttcggaagctagcgggtcgtgctctcgggaagctgcaccgccttctggagaagcggcaggaaggcgcggagacgctggagctgagtgcggatgggcgcccggtgaccacgcagacccgggacccgccggtggtggactgcacctgcttcggcctccctcgccgctacattatcgccatcatgagtggtctgggcttctgcatcagctttggcatccgctgcaacctgggcgtggccatcgtctccatggtcaataacagcacgacccaccgcgggggccacgtggtggtgcagaaagcccagttcagctgggatccagagactgtcggcctcatacacggctcctttttctggggctacattgtcactcagattccaggaggatttatctgtcaaaaatttgcagccaacagagttttcggctttgctattgtggcaacatccactctaaacatgctgatcccctcagctgcccgcgtccactatggctgtgtcatcttcgtgaggatcctgcaggggttggtagagggggtcacataccccgcctgccatgggatctggagcaaatgggccccacccttagaacggagtcgcctggcgacgacagccttttgtggttcctatgctggggcggtggtcgcgatgcccctcgccggggtccttgtgcagtactcaggatggagctctgttttctacgtctacggcagcttcgggatcttctggtacctgttctggctgctcgtctcctacgagtcccccgcgctgcaccccagcatctcggaggaggagcgcaagtacatcgaggacgccatcggagagagcgcgaaactcatgaaccccctcacgaagtttagcactccctggcggcgcttcttcacgtctatgccagtctatgccatcatcgtggccaacttctgccgcagctggacgttctacctgctgctcatctcccagcccgactacttcgaagaagtgttcggcttcgagatcagcaaggtaggcctggtgtccgcgctgccccacctggtcatgaccatcatcgtgcccatcggcggccagatcgcggacttcctgcggagccgccgcatcatgtccaccaccaacgtgcgcaagttgatgaactgcggaggcttcggcatggaagccacgctgctgttggtggtcggctactcgcactccaagggcgtggccatctccttcctggtcctagccgtgggcttcagcggcttcgccatctctgggttcaacgtgaaccacctggacatagccccgcgctacgccagcatcctcatgggcatctccaacggcgtgggcacactgtcgggcatggtgtgccccatcatcgtgggggccatgactaagcacaagactcgggaggagtggcagtacgtgttcctaattgcctccctggtgcactatggaggtgtcatcttctacggggtctttgcttctggagagaagcagccgtgggcagagcctgaggagatgagcgaggagaagtgtggcttcgttggccatgaccagctggctggcagtgacgacagcgaaatggaggatgaggctgagcccccgggggcaccccctgcacccccgccctcctatggggccacacacagcacatttcagccccccaggcccccaccccctgtccgggactactgaccatgtgcctcccactgaatggcagtttccaggacctccattccactcatctctggcctgagtgacagtgtcaaggaaccctgctcctctctgtcctgcctcaggcctaagaagcactctcccttgttcccagtgctgtcaaatcctctttccttcccaattgcctctcaggggtagtgaagctgcagactgacagtttcaaggatacccaaattcccctaaaggttccctctccacccgttctgcctcagtggtttcaaatctctcctttcagggctttatttgaatggacagttcgacctcttactctctcttgtggttttgaggcacccacaccccccgctttcctttatctccagggactctcaggctaacctttgagatcactcagctcccatctcctttcagaaaaattcaaggtcctcctctagaagtttcaaatctctcccaactctgttctgcatcttccagattggtttaaccaattactcgtccccgccattccagggattgattctcaccagcgtttctgatggaaaatggcgggaattcctgcagcccgggggatccact

SEQ ID NO: 4 - protein, 560 aa, Homo sapiens
MEFRQEEFRKLAGRALGKLHRLLEKRQEGAETLELSADGRPVTTQTRDPPVVDCTCFGLPRRYIIAIMSGLGFCISFGIRCNLGVAIVSMVNNSTTHRGGHVVVQKAQFSWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPLTKFSTPWRRFFTSMPVYAIIVANFCRSWTFYLLLISQPDYFEEVFGFEISKVGLVSALPHLVMTIIVPIGGQIADFLRSRRIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDDSEMEDEAEPPGAPPAPPPSYGATHSTFQPPRPPPPVRDY

SEQ ID NO: 5 - DNA, 2024 bp, Rattus norvegicus
gaattcggcacgagcggagctgcggggccgggccgggccggggcggaccccgggatcccggacgcggccgcccgggcccgcgggcggggggattggcaggggacccgcgtgggcacagccaccatggagttccggcaggaggagtttcggaagctggcggggcgcgccctggggaggctgcaccggttactggagaagcggcaggaaggcgcggagacattggagctgagcgccgacgggcgcccagtgaccacacacacgcgggacccgccggtggtggactgcacttgctttggcctccctcgccgctacatcatcgcgatcatgagcggtctgggtttctgcatcagctttggcatccgctgcaacctgggcgtggccatcgtatccatggtcaacaacagtacaacccaccgtgggggccacgtggtggtgcagaaagcccagttcaactgggatccagagactgtcggcctcatacatggctcctttttctgggggtacattgtcactcagattcctggaggatttatctgccaaaaattcgcagccaacagggtctttggctttgccattgtggctacctccaccctaaatatgttgatcccttcagcagcccgtgttcactatggctgtgtcatcttcgtgaggatccttcagggattggtggagggggtcacataccctgcttgccatggcatctggagcaaatgggcccctcccttagaacggagtcggctggcgacgacagccttttgcggttcctatgccggggcagtggttgccatgcctctggctggggtcctggtacagtattcaggatggagttctgtcttctatgtctatggcagcttcgggatcttttggtacctgttctggttgcttgtctcctacgagtcacctgcactacaccccagcatctccgaggaggagcgcaaatacattgaggatgccatcggagaaagcgccaagctcatgaaccctgttacgaagtttaacacaccctggaggcgcttctttacctccatgccggtctatgccatcattgtcgccaacttttgccgcagctggactttctacctgctcctcatctcccagcccgcctactttgaagaagtgttcggctttgagatcagcaaggtgggactggtgtcggcactgcctcaccttgtcatgactatcatcgtacccatcggaggccagatcgccgacttcctgcgcagtcgtcatataatgtccacgaccaatgtgcgaaagctgatgaactgcgggggtttcgggatggaagctacgctgctgctggtggtcggatactcacactccaagggcgtggccatctccttcctggtcctggctgtgggcttcagtggctttgctatctctgggtttaacgtgaaccacttggacatcgcccctcgatatgccagcatcttgatgggcatttccaatggcgtgggcacactgtctgggatggtgtgccccatcatcgtgggtgcaatgaccaagcacaagacgcgggaggagtggcagtacgtgttcctcatagcctccctggtgcactatggaggtgtcatcttctatggggtctttgcttcgggagagaaacagccgtgggcagagccggaggagatgagcgaggagaagtgtggctttgttggccacgaccagctggctggcagtgacgaaagtgaaatggaagacgaggttgagcccccgggggcaccccccgcacctccgccttcctacggggccacacacagcacagttcagcctccaaggcccccaccccctgtccgggactactgaccacgtgcctcccactggtgggcagtttccaggacctccactcgatacacctctagcctaaacggcagtgtcgaggaaccccactcctctcctgcctcaggcttaagatgcaagtcttcccttgtgcccagtgctgtccgaccagccctctctccttctcaactgcctcttgcaggggtgaagctgcacactagcagtttcaagctcgtgccgaattc

SEQ ID NO: 6 - protein, 560 aa, Rattus norvegicus
MEFRQEEFRKLAGRALGRLHRLLEKRQEGAETLELSADGRPVTTHTRDPPVVDCTCFGLPRRYIIAIMSGLGFCISFGIRCNLGVAIVSMVNNSTTHRGGHVVVQKAQFNWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPVTKFNTPWRRFFTSMPVYAIIVANFCRSWTFYLLLISQPAYFEEVFGFEISKVGLVSALPHLVMTIIVPIGGQIADFLRSRHIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDESEMEDEVEPPGAPPAPPPSYGATHSTVQPPRPPPPVRDY

SEQ ID NO: 7 - DNA, 2836 bp, Mus musculus
cggccgcccgggcccgcgggcggggggattggcaggggacccgcgtgggcacagccaccatggagttccggcaggaggagtttcggaagctggcggggcgcgccctggggaggctgcaccggttactggagaagcggcaggaaggcgcggagacactggagctgagtgccgacgggcggccagtgaccacgcacactcgggacccgcctgtggtggactgcacctgctttggcctccctcgtcgctacatcatcgccatcatgagcggtctgggtttctgtatcagctttggcatccgctgcaacctgggcgtggccatcgtgtccatggtcaacaacagcacaacccaccgtgggggccacgtggtggtgcagaaagcccagttcaactgggatccagagactgtcggcctcatacatggctcctttttctggggctacattgtcactcagattcctggaggatttatctgccaaaaattcgcagccaacagggtctttggctttgccattgtggctacctccaccctaaacatgttgatcccttcagcagcccgcgttcactatggctgtgtcatcttcgtgaggatccttcagggattggtggagggggtcacataccctgcttgccatggcatctggagcaaatgggcccctcccttagaacggagtcggctggcaacgacagccttttgcggttcctatgctggggcggtggttgccatgcccttggctggggtccttgtgcagtattcaggatggagttctgtcttctatgtctatggcagcttcgggatcttttggtacctgttctggttgcttgtctcctatgagtcaccggcactgcaccccagcatctctgaggaggagcgcaaatacattgaggatgccatcggggagagcgccaagctcatgaaccctgttacgaagtttaacacaccctggaggcgcttctttacgtccatgcccgtctatgccatcatcgttgcgaacttttgccgcagctggaccttctacctgctcctcatctctcagcccgcctactttgaagaagtgttcggctttgagatcagcaaggtggggctggtgtcggcgctgcctcaccttgtcatgaccatcatcgtacccattggaggccagatcgctgactttttgcgcagtcgtcacataatgtccactaccaacgtgcgaaagctcatgaactgcgggggtttcgggatggaagccacgctgctgctggtggtcggatactcgcactccaagggcgtggccatctccttcctggtcctggctgtgggcttcagtggctttgccatctctgggtttaacgtgaaccacttggacatcgcccctcgctatgccagcatcttgatgggcatttccaatggcgtgggcacactgtctgggatggtgtgccccatcatcgtgggtgcaatgaccaagcacaagacgcgggaggagtggcagtacgtgttcctcatagcctccctggtgcactacggcggtgtcatcttctatggggtctttgcttcgggagagaagcagccgtgggcagagccggaggagatgagcgaggagaagtgtggctttgttggccacgaccagctggctggcagtgacgaaagtgaaatggaggacgaggctgagcccccaggggcgccccccgcgccgcctccgtcctacggggccacacacagcacagtgcagcctccgaggcccccgccccctgtccgggactactgaccacgggcctcccactgtggggcagtttccaggacttccactccatacacctctagcctgagcggcagtgtcgaggaaccccactcctcccctgcctcaggcttaagatgcaagtcctcccttgttcccagtgctgtccgaccagccctctttccctctcaactgcctcctgcggggggtgaagctgcacactagcagtttcaaggatacccagactcccctgaaagtcgttctccgcttgtttctgcctgtgtgggctcaaatctcccctttgagggctttatttggagggacagttcaacctcttcctctcttgtggttttgaggtttcaccccttcccccaagaccccagggattctcaggctaccccgagattattcaggtggtcccctactcagaagacttcatggtcgtcctctattagtttcaaggctcgcctaaccaattctacatttttccaagctggtttaacctaaccaccaatgccgccgttcccaggactgattctcaccagcgtttctgagggaaaatggcggtttcaagtccccccaccccccttttcttccctcgtcccctcaccagcacactttgccgggccttgaccttagcttagtacaatcattgtccagggaaatggccaaaatggctctgctcaccccgtgctctttttctgactcagttttcaggtctcagtagtggctgcccaaagctattaattcagcggctcgaggccacctcttcctccccgtggtggtttcaggatccccctcgcccccccccccccaaatccttgcactttattctcctgggtggttccaggccgccctcggtttctcagtggccatttgttgtgtgtccctcaggggctaaatgattccaaatctggggtgcttcccctcatagacacccctctctcaacgtagaaatctgggtgggggtgaggtgtgtgagagaagttacagaatcccaggaaagggagcggggctgggaggagagggttgttcctggggcagggtcgtgtcttggtgtctgtctctgtgacgtaaatcctgccctgccccaccccactcccaataaacgctctggtgtacgg

SEQ ID NO: 8 - protein, 560 aa, Mus musculus
MEFRQEEFRKLAGRALGRLHRLLEKRQEGAETLELSADGRPVTTHTRDPPVVDCTCFGLPRRYIIAIMSGLGFCISFGIRCNLGVAIVSMVNNSTTHRGGHVVVQKAQFNWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPVTKFNTPWRRFFTSMPVYAIIVANFCRSWTFYLLLISQPAYFEEVFGFEISKVGLVSALPHLVMTIIVPIGGQIADFLRSRHIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDESEMEDEAEPPGAPPAPPPSYGATHSTVQPPRPPPPVRDY

SEQ ID NO: 9 - DNA, 3946 bp, Homo sapiens
cgtttaaaagccatcagatttgagagcaataagtcttcaaaaccgggaatttacattgtttttcagctgaccgacttccaggaaaaggactcaaccgcatctacccaaataccgtggcactgcttgcgctctttgccaccggatactccccttccaatgagactttctgattgtgtctaccaactctcctattaggaaacccgtgggttgcatgcagctattctgttgtattctcattctcactctccctcccttctctcactctcactcttgctggaggcgagccactaccattctgctgagaaggaaaagcccgcaactactttaagagattaagacaatatgcgcaatcctcgcctttcctagcaatcactatttaaatctggcaagaactgacaacagtctttgcaagaatggaatccgtaaaacaaaggattttggccccaggaaaagaggggctaaagaattttgctggaaaatcactcggccagatctacagggtgctggagaagaagcaagacaccggggagacaatcgagctgacggaggatgggaagcccctagaggtgcccgagaggaaggcgccgctgtgcgactgcacgtgcttcggcctgccccgccgctacattatcgccatcatgagcggcctgggcttctgcatctccttcggtatccgctgcaacctgggcgtggccattgtggacatggtcaacaacagcaccatccaccgcgggggcaaggtcatcaaggagaaagccaaattcaactgggacccggaaaccgtggggatgatccacggttccttcttttggggctacatcatcactcagattccgggaggctacatcgcgtctcggctggcagccaacagggttttcggagctgccatacttcttacctctaccctaaatatgctaattccatcagcagccagagtgcattatggatgtgtcatctttgtcagaatactgcagggacttgttgagggtgtgacctacccagcatgtcatgggatatggagcaaatgggccccacctctagagaggagtagactggcaaccacctccttttgtggttcctatgccggagctgtgattgcaatgcctttagctggcattcttgtgcagtacactggctggtcttcagtgttttatgtctacggaagctttggaatggtctggtacatgttttggcttttggtgtcttatgaaagtcctgcaaagcatcctactattacagatgaagaacgtaggtacatagaagaaagcattggagagagtgcaaatcttttaggtgcaatggaaaaattcaagactccatggaggaagttttttacatccatgccagtctatgcaataattgttgcaaacttctgcagaagctggactttttatttattgcttattagtcagccagcatattttgaggaagtctttggatttgaaattagcaaggttggtatgctatctgctgtgccacacttagtaatgacaattattgtgcctattgggggacaaattgcagattttctaagaagcaagcagattctttcaactacgacagtgagaaagatcatgaattgtggtggttttggcatggaagccacactgctcctggtcgttggctattctcatactagaggggtagcaatctcattcttggtacttgcagtgggattcagtggatttgctatatctggtttcaatgttaaccacttggatatcgctccaagatatgccagtatcttaatgggcatttcgaatggtgttggcacattgtcaggaatggtttgtcctatcattgttggtgcaatgacaaagaataagtcacgtgaagagtggcagtatgtcttcctgatcgctgccctagtccactatggtggagttatattttatgcaatatttgcctcaggagagaaacaaccctgggcagacccggaggaaacaagtgaagaaaaatgtggatttattcatgaagatgaactcgatgaagaaacaggggacattactcaaaattatataaattatggtaccaccaagtcttatggtgccacaacacaggccaatggaggttggcctagtggttgggaaaagaaagaggaatttgtacaaggagaagtacaagactcacatagctataaggaccgagttgattattcataacaaaactaattactggatttatttttagtgtttgtgattaaattcattgtgattgcacaaaaattttaaaaacacgtgatgtaaacttgcaagcatatcaaccaggcaagtcttgctgtaaaaatgaaaacaaaacaaacccatgaggttaccatcaagtgcaatctgtaaaattgtgaagttccatcatttccattcaagtcatccattcttgcatttgtgacttaaaggttgactggtcaaaattgtagaaacaagtagttacccattggattcatatgagctaaaactcatcactatttactaaagcacaacatctcatcctacaaaagttaagaagccaaagctacttgatcatgcaaaatgcacttatatatttgttacactgtattgcaagatagcacacagaagttggctgcgtcaagtagaggcgacatttattaagtgaaaatcatggagttgggatatctctcaattaaagaaatacattgtgaactatcagctacaaagttgtactgaataactattagaattgcataatgtgagatattttgttagtcctcaaaaggaatatcttgcagtgttttctatgaaatgcttgggcacaaacacttatttctgtgaaagagaacatgtaagttgaggggtatgcttcatgttcttccatccatttacctaatagtatgaaacagttcacatttcaataaaatcaaacttttcatgtagcgtatcacataacttttttgcaaaaaatataaaaagaaataaacttcaatgtattttttattacaactttgtactggttgtaacttgcattagaaaaaaaaaagagatatataaaccacaaagaatctaataagaaatttattatggagatatagcccttaaaatgcaatattaagaacaaagaaatagaaaatggtttagatatctttcttccttcataattaaatactatatgaaacttgtgccacagagctatatgtaatatgaaaagattaacttcatagagatattgtaagtaggtaattttattatttaaagtcctattaagaaatatttgtcttaaatatataggacaatacattatattaaaatggtctctctctatatatatctgtatatcttatacatgtccatacacagaaacataataaacaatcttcacacgaaaccaaaaatagcatacacctaatgttgggttagggaattgcaatttctactttcatagagtcatagaattttaggtggggaagaggcattttgcttgtcatttcttaatataactcaacaagaattgcaacatttgtgtaccaagcaataagtgcaatgcataaaatttcctgtctgtatattaccttcattttgcttgtagtagctgtttgggtggttggaataattttatttttcttttaaaaaagctaacatcagacccctttataatgtcctaaaattatgataatacatttcccaattcaactcaaaatattattggtgtattttgtctattctggatatttgatctgtttaatgtactgtgctagtgactggaggccctgctactgcaaatataaaacctaaagtttgtttaaaaaaatgcaaatcattctttaccttaagaaaaaaaaaataccctttgctttgtgcctcaaagtgatgtaatgtgatcacagcttttgttgtgttgaatgaaaatatgtggactgtcattttgttgcagcaaaaaagtgttaataaaatgctctatttatccttttttaaaaaaaaaaaaaaaaaaaaaaaaa

SEQ ID NO: 10 - protein, 582 aa, Homo sapiens
MESVKQRILAPGKEGLKNFAGKSLGQIYRVLEKKQDTGETIELTEDGKPLEVPERKAPLCDCTCFGLPRRYIIAIMSGLGFCISFGIRCNLGVAIVDMVNNSTIHRGGKVIKEKAKFNWDPETVGMIHGSFFWGYIITQIPGGYIASRLAANRVFGAAILLTSTLNMLIPSAARVHYGCVIFVRILQGLVEGVTYPACHGIWSKWAPPLERSRLATTSFCGSYAGAVIAMPLAGILVQYTGWSSVFYVYGSFGMVWYMFWLLVSYESPAKHPTITDEERRYIEESIGESANLLGAMEKFKTPWRKFFTSMPVYAIIVANFCRSWTFYLLLISQPAYFEEVFGFEISKVGMLSAVPHLVMTIIVPIGGQIADFLRSKQILSTTTVRKIMNCGGFGMEATLLLVVGYSHTRGVAISFLVLAVGFSGFAISGFNVNHLDIAPRYASILMGISNGVGTLSGMVCPIIVGAMTKNKSREEWQYVFLIAALVHYGGVIFYAIFASGEKQPWADPEETSEEKCGFIHEDELDEETGDITQNYINYGTTKSYGATTQANGGWPSGWEKKEEFVQGEVQDSHSYKDRVDYS

SEQ ID NO: 11 - DNA, 3982 bp, Rattus norvegicus
agacagtaaggttcttttgcttttttcccttacacaaggattcgatgacgtttttggtcaatctgattaaaagacagcggatttggttgcgttaagacttcaaaaccgggaatttacgttgtttttcggtgaggtgacttccagaacggggactcatcagcacccgcccaaataccacggcactgcgcgcgccctcggccaccggatcctccccttccaatgagactttgtgactgtgtgtaccaattctcctattaggaaacccgtgggctgaatgcagctattccgttgtactctctttctcgctctccctcccctctccaactcacagccttgctgaaaagctcatctctgctgagaagaaaacgttctaccttaacctattaagactatgcgcagaactcgcctttcatagccatcacaatttaaatctggtaaggctggacacgagtctttacaagaatggagtcggtaaaacaaaggattttggccccggggaaagaggggataaagaattttgctggaaaatccctcggacagatctacagggtgctggagaagaagcaggataaccgagagaccatcgagctgacagaggacggcaagcccctggaggtgcctgagaagaaggctccgctatgcgactgtacgtgcttcggcctgccgcgccgctacatcatagccatcatgagcggcctcggcttctgcatctcctttggtatccgctgtaacctgggtgtggccattgtggacatggtcaacaacagcaccatccaccggggtggcaaagttatcaaggagaaagccaagtttaactgggaccccgagactgtggggatgattcacgggtcgttcttctggggctatatcatcacgcagattccgggcggatacatcgcatcgcgactggctgctaaccgggtctttggggctgccatactgcttacctctaccctcaatatgctgatcccatctgcagccagagtgcattatggatgcgtcatctttgttagaatattgcaaggacttgtggagggcgtcacctacccagcctgtcacgggatatggagcaagtgggcccctcctttggagaggagtaggttggctaccacctccttctgtggttcctatgctggagcagtcattgcaatgcccctagctggtatcctggtgcagtacactggatggtcttcagtattttacgtatatggaagctttggtatggtctggtatatgttctggcttctggtgtcttacgagagccccgcaaagcatccaaccataacagacgaagaacgtaggtacatagaagagagcatcggggagagcgcaaatctgttaggagcaatggagaaattcaagaccccatggaggaagtttttcacatccatgcccgtctatgcgataattgttgcaaacttctgcaggagttggactttttatttactgctcatcagtcaaccagcttatttcgaggaggtttttggatttgaaatcagcaaggttggcatgttgtctgcggtcccacacctggtcatgacaatcattgtgcctatcggggggcaaattgcagactttctaaggagcaagcaaattctttcaacaactacagtgcgaaagatcatgaactgcgggggttttggcatggaagccacactgcttctggttgttggctactctcatactagaggggtggccatctccttcttggtgcttgcagtgggattcagtggatttgctatctctggtttcaatgtgaaccacttggatattgccccgagatatgccagtatcttaatgggcatttcaaatggtgttggcacgctgtcgggaatggtctgcccgatcattgttggtgcaatgacgaagaacaagtcccgtgaagaatggcagtatgtcttcctcatcgctgcactggtccactatggtggagtcatattttatgcactatttgcctcaggagagaagcaaccttgggcagaccctgaggaaacaagcgaagaaaagtgtggcttcattcatgaagatgaactggatgaagaaacgggggacatcactcagaattacataaattacggtaccaccaaatcctacggcgccacctcacaggagaacggaggctggcctaacggctgggagaaaaaggaagaatttgtgcaagaaagtgcgcaagacgcgtactcctataaggaccgagatgattattcataacgaagctagttgctggattcctttgtagtgtttgtgattaaattaattgtgattgcacaaaatcattttaagaaatgtggtgtaaacatgtaaacacatcaaccaagcaagtcttgctgttcaaaaaataataataatatgaattcaaaacagaccgtgagagtcccatcaagtgcaatctgtggcggcagtcacgtgacgccatttccattcaggccattcgtccttttcgtttgtgatttaaaggtttcctgtagaaataagtaggtattcgttggatccatcaccacgttagagagtacaactacaacagttggcacatgtcatcctacggaagttaggaagccaaagctactggattatgtgaactgcatttatttatttattacactggactgcaaaatatcccagggaaatcctgtctagagacatagtagaactggaaagatggctagattgggtactgacgataatcattgtgtgtatatcatggagtggctatatcttttaattggagaactatattgtatagctagcaaaattgtactgaattattactaggagtgcacagtgtgtgatattttgtgatcttccaaaagcttatcttgcagtgttttgtgaaacgcttgggcacaaacacttatttttatgaacaagagcttgtaaagggaggagtatgctccatgctctcccattcactacctgacagtatcaaaccttcacatttcaatgaaatccaacgtccatgtaacatatcacatgactttttttgcaaaaaagaatataagaagaaatagacttcaatgtattttttattacaactttgtactggttgtaacttgcattaggaaaaatgattaatatatgtataatcgtaaagaatctaataaaaatttactatgaagatatagcccttaaaatgcaatattaacaacaaaaatatatagaaaatttagataatcttccttgataactagagactatatggaactcacaccacaaagctatatataatatgaaaagataaacaatagagattgtatatgtagacgattttatgacctaatgtcccatttaagaggtatttgtcttgagtatatagtacaaagtatattaaaattatatctacatccctgtatatcttatacatatccactcacacaaacataacaaatacttttcacacagaaccaaaaacaagcatacacctaatgttgggtttggggattgcaatttctactttcatagagtcatagaattttagatgggaaaaaaaaaggcattttgctcgtcatttcttaatataattaattcaacaggaactgcaacatttgtgtaccaagcaataagtgcgaagcataaacctgctgtgtgtaaactatccccatactgcttgtggtagcactgatttctttcttttaaagaacttaacatcggagctctttacaatgttttgcgctgataagaatgcacatcccaatttaacgcaaagtgtcacctggtgtgtttacctgtctgttttgggtatttggtctgtttggtgtcctgtgctcttgactggaggccctgctactgcgaatataaaacgtgaagtttgtttctaaatgcaaaccactcctgaccttaagaaactaaagtccctctctgctttgtgtctccaagtactatcatgtgaccataacccttgctgtgctgagtaaaaagatgtgaactgtcattttgttgctgcgaagcaagtgttaataaaatgttctatttaaaaaaaaaaaaa

SEQ ID NO: 12 - protein, 582 aa, Rattus norvegicus
MESVKQRILAPGKEGIKNFAGKSLGQIYRVLEKKQDNRETIELTEDGKPLEVPEKKAPLCDCTCFGLPRRYIIAIMSGLGFCISFGIRCNLGVAIVDMVNNSTIHRGGKVIKEKAKFNWDPETVGMIHGSFFWGYIITQIPGGYIASRLAANRVFGAAILLTSTLNMLIPSAARVHYGCVIFVRILQGLVEGVTYPACHGIWSKWAPPLERSRLATTSFCGSYAGAVIAMPLAGILVQYTGWSSVFYVYGSFGMVWYMFWLLVSYESPAKHPTITDEERRYIEESIGESANLLGAMEKFKTPWRKFFTSMPVYAIIVANFCRSWTFYLLLISQPAYFEEVFGFEISKVGMLSAVPHLVMTIIVPIGGQIADFLRSKQILSTTTVRKIMNCGGFGMEATLLLVVGYSHTRGVAISFLVLAVGFSGFAISGFNVNHLDIAPRYASILMGISNGVGTLSGMVCPIIVGAMTKNKSREEWQYVFLIAALVHYGGVIFYALFASGEKQPWADPEETSEEKCGFIHEDELDEETGDITQNYINYGTTKSYGATSQENGGWPNGWEKKEEFVQESAQDAYSYKDRDDYS

SEQ ID NO: 13 - DNA, 2528 bp, Mus musculus
ggcacgaggctcagtcttaattccactctgccactctgccgcagagcacaattacgccggcgcgatgggaggagaccatgttaaggcaggaagctaacagcagccgctcacctgaggcctaggaagctcccaagggttctgagagctatgagctctgatcagcaaagtcaccattttagacagtagggttcttttgcttttttccttacacaagggttcgatgacgtttctggtcaatctgattaaaagacagcggatttgattgcgataagacttcaaaaccgggaatttacgttgtttttcggtgaggtgacttccagaacagggactcatcagcacccgcccaaataccacggcactgcgcgcgccctcggccaccggatcctccccttccaatgagactttgtgactgtgtgtaccaattctcctattaggaaacccgtgggctgcatgcagctattctgttgtactctctttctcgccctccctcccctctccaactcacagccttgctggaaagctcacctctgctgagaagaaaaagctctaccttaaccaactaagactatgcgcagaatccgtctttcatagccacaacaatttaaatctggtaaggctggacaccagtctttacaagaatggagtcggtaaaacaaaggattttggccccggggaaagaggggataaagaattttgctggaaaatccctcggacagatctacagggtgctggagaagaagcaggacaaccgagagaccatcgagctgacagaggacggtaagcccctggaggtgcctgagaagaaggctccgctatgcgactgcacgtgcttcggcctgccgcgccgctacatcatagccatcatgagcggcctcggcttctgcatatccttcggcatccgctgtaacctgggcgtggccatcgtggacatggtcaacaacagcactatccaccgcggaggcaaagttatcaaggagaaagccaaatttaactgggaccccgagaccgtggggatgatccacggatcgttcttctggggctatatcatcacccagattccaggaggatatatcgcatcgcggctggctgctaaccgggtctttggggctgcgatactgctcacctctaccctcaatatgctgatcccatctgcagccagagtgcattatggatgtgtcatctttgttaggatattgcaaggacttgtggagggtgtcacctacccagcctgtcatgggatatggagcaagtgggcccctcccttggagaggagtaggttggctacaacctccttttgtggttcctatgctggagcagtcattgcaatgcccttagctggtatccttgtgcagtacactggatggtcgtcagtattttatgtgtatggaagctttggcatggtctggtacatgttctggcttctggtgtcttatgagagccctgcaaagcatcctaccattacagatgaagaacgtaggtacatagaggagagcattggagagagcgcaaatctgctaggtgcaatggaaaaatttaagaccccatggaggaagtttttcacatccatgcccgtctacgcgataattgttgccaacttctgcaggagctggactttttatttactgctcatcagtcagccagcttattttgaggaggtttttggatttgaaatcagcaaggttggcatgttgtctgcagtccctcaccttgtcatgacaatcattgtgcctatcggggggcaaattgcagatttcctaaggagcaagcaaattctctcaacaactacagtgagaaagatcatgaattgtgggggttttggcatggaagccacgctgcttctggttgttggctactctcatactagaggggtggccatctccttcttggtgcttgcagtaggattcagtggatttgctatctctggtttcaatgttaatcacttggatattgctccaagatatgccagtatcttaatgggcatttcaaatggcgttggcacgctgtcggggatggtttgccctatcattgttggtgcaatgacaaagaataagtcccgtgaagaatggcagtatgtcttcctcattgctgcactcgtccactatggtggagtcatattttatgcactatttgcctcaggagagaaacaaccttgggcagaccctgaggaaacaagcgaagaaaaatgtggcttcattcacgaagatgaactggatgaagaaacgggggacatcactcagaattacataaattacggtaccaccaaatcttacggtgctacctcacaggagaatggaggctggcctaacggctgggagaaaaaggaagaatttgtgcaagaaggtgcgcaagacgcgtacacctataaggaccgagatgattattcataacgatgctagttgctggattcatttgtagtgtttgtgaatcaattaattgtgattgcacaaaaataattttaaaaatgtggtgtgaacatgtaaacatatcaaccaagcaagtcttgctgttcaaaaaaaaaaaaaaaaaaa

SEQ ID NO: 14 - protein, 582 aa, Mus musculus
MESVKQRILAPGKEGIKNFAGKSLGQIYRVLEKKQDNRETIELTEDGKPLEVPEKKAPLCDCTCFGLPRRYIIAIMSGLGFCISFGIRCNLGVAIVDMVNNSTIHRGGKVIKEKAKFNWDPETVGMIHGSFFWGYIITQIPGGYIASRLAANRVFGAAILLTSTLNMLIPSAARVHYGCVIFVRILQGLVEGVTYPACHGIWSKWAPPLERSRLATTSFCGSYAGAVIAMPLAGILVQYTGWSSVFYVYGSFGMVWYMFWLLVSYESPAKHPTITDEERRYIEESIGESANLLGAMEKFKTPWRKFFTSMPVYAIIVANFCRSWTFYLLLISQPAYFEEVFGFEISKVGMLSAVPHLVMTIIVPIGGQIADFLRSKQILSTTTVRKIMNCGGFGMEATLLLVVGYSHTRGVAISFLVLAVGFSGFAISGFNVNHLDIAPRYASILMGISNGVGTLSGMVCPIIVGAMTKNKSREEWQYVFLIAALVHYGGVIFYALFASGEKQPWADPEETSEEKCGFIHEDELDEETGDITQNYINYGTTKSYGATSQENGGWPNGWEKKEEFVQEGAQDAYTYKDRDDYS